(19) United States
(12) Patent Application Publication
(10) Pub. No.: US 2023/0241063 A1
(43) Pub. Date: Aug. 3, 2023

(54) METHODS OF TREATING PROSTATE CANCER

(71) Applicant: Celgene Quanticel Research, Inc., San Diego, CA (US)

(72) Inventors: JUAN DE ALVARO, Summit, NJ (US); JOSEP LLUIS PARRA-PALAU, Summit, NJ (US); ZARIANA NIKOLOVA, Summit, NJ (US); JORGE DI MARTINO, Summit, NJ (US); ELLEN FILVAROFF, Summit, NJ (US); IDA ARONCHIK, Summit, NJ (US); MARTINA MALATESTA, Summit, NJ (US)

(73) Assignee: Celgene Quanticel Research, Inc., San Diego, CA (US)

(21) Appl. No.: 18/007,925

(22) PCT Filed: Jun. 4, 2021
(86) PCT No.: PCT/US2021/035863

§ 371 Date: Dec. 2, 2022

Related U.S. Application Data
(60) Provisional application No. 63/035,622, filed on Jun. 5, 2020.

(51) Int. Cl. A61K 31/513; A61K 31/58; A61P 35/04; A61K 9/00; A61K 31/573

(52) U.S. Cl. CPC A61K 31/513; A61K 31/58; A61P 35/04; A61K 9/0053; A61K 31/573

(57) ABSTRACT

The present application relates generally to methods for treating prostate cancer with a lysine specific demethylase-1 (LSD-1) inhibitor, wherein the lysine specific demethylase-1 (LSD-1) inhibitor resensitizes the prostate cancer cells to androgen receptor pathway inhibitor (ARPI) treatment.

### CROSS-REFERENCE TO RELATED APPLICATION

The present application claims priority to U.S. Provisional Patent Application No. 63/035,622, filed Jun. 5, 2020, the contents of which are specifically incorporated herein by reference.

### FIELD

The present application relates generally to compositions and methods for treating prostate cancer with a lysine specific demethylase-1 (LSD-1) inhibitor, wherein the lysine specific demethylase-1 (LSD-1) inhibitor resensitizes the prostate cancer cells to androgen receptor pathway inhibitor (ARPI) treatment.

### BACKGROUND

Prostate cancer is the second leading cause of cancer-related death and the most commonly diagnosed cancer in men. Prostate cancer tumors are composed primarily of prostate luminal epithelial cells. Differentiation of prostate luminal epithelial cells is controlled in part by Androgen receptor (AR)-driven expression of prostate-specific markers. AR are steroid receptors which function as transcription factors that control survival of the cells through mechanisms that remain unclear. Depletion of androgens causes death of normal prostate luminal epithelial cells, which demonstrates the critical role of the AR pathway in their survival. Cancerous prostate cells continue to express AR and their survival also depends on the presence of androgens, which makes androgen deprivation the therapy of choice for patients with advanced prostate cancers. First-line treatments for prostate cancer aim to reduce circulating androgen levels through the use of androgen deprivation therapies (ADT). While ADT is initially effective at reducing prostate cancer growth, after two to three years of treatment, the majority of patients progress to castration-resistant prostate cancer (CRPC) and tumor growth will proceed even in the presence of castrate levels of androgen. At this point of disease progression, the number of therapeutic options becomes very limited.

Almost all types of prostate cancers are adenocarcinomas. These cancers develop from the prostate gland cells, the cells that make the prostate fluid that is added to semen. There are other types of cancers that originate in the prostate, and some are defined by the tissue in which they originate and others are defined by their resistence to therapies.

Castrate-resistant prostate cancer (CRPC) is defined by disease progression despite androgen depletion therapy (ADT) and may present as either a continuous rise in serum prostate-specific antigen levels, the progression of pre-existing disease, and/or the appearance of new metastases. Potent hormone therapies like abiraterone and enzalutamide can be effective treatments for men with castrate-resistant prostate cancer (CRPC). However, almost all men eventually develop drug resistance to these agents.

Neuroendocrine cells are one of the epithelial populations in the prostate. Neuroendocrine prostatic cancer (NEC) is considered as a special type of neuroendocrine differentiation of prostatic epithelial neoplasms. The definition of neuroendocrine prostatic carcinomas is still emerging and includes a variety of subtypes.

Thus, there remains a need for more effective treatments for prostate cancer, and this disclosure satisfies this need.

### SUMMARY

The present application relates generally to compositions and methods for treating prostate cancer. The methods comprise administering a lysine specific demethylase-1 (LSD-1) inhibitor, or a pharmaceutically acceptable salt thereof, wherein the lysine specific demethylase-1 (LSD-1) inhibitor resensitizes the prostate cancer cells to androgen receptor pathway inhibitor (ARPI) treatment. In some embodiments, these methods comprise administering a LSD-1 inhibitor, or a pharmaceutically acceptable salt thereof, with an androgen receptor pathway inhibitor (ARPI), or a pharmaceutically acceptable salt thereof.

The aspects and embodiments of the present disclosure provide for methods and pharmaceutical compositions for treating subjects with prostate cancer, such as castration resistant prostate cancer (CPRC), neuroendocrine prostate cancer (NEPC), anti-androgen resistant prostate cancer, enzalutamide resistant prostate cancer, abiraterone resistant prostate cancer, ARPI induced drug resistant prostate cancer. In some embodiments, the prostate cancer is metastatic prostate cancer. At least one embodiment provides a method for treating prostate cancer where administration of the ARPI enhances the therapeutic effect by resensitizing prostate cancer cells to the ARPI.

Provided in one aspect is a method of treating metastatic castration-resistant prostate cancer (mCRPC) in a subject in need thereof comprising administering to the subject a besylate salt of an LSD-1 inhibitor compound having the structure:

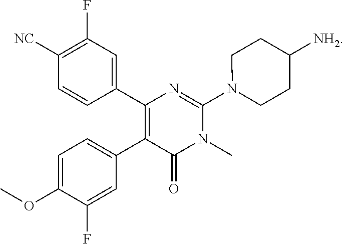 [SMILES: COc1ccc(-c2c(-c3ccc(C#N)c(F)c3)nc(N3CCC(N)CC3)n(C)c2=O)cc1F]

In some embodiments, in a first 28-day cycle, the LSD-1 inhibitor is administered (a) orally; and/or (b) at the dose of about 60 mg; and/or (c) once a week; and/or (d) on Days 1, 8, 15 and 22 of a 28 day cycle.

In some embodiments, the subject has failed prior ARPI therapy. In some embodiments, the prior ARPI therapy is enzalutamide.

In some embodiments, the method further comprises the step of administering an ARPI and a corticosteroid, such as prednisone, in combination with the LSD-1 inhibitor. In some embodiments, the subject has failed prior ARPI therapy and the ARPI is either different or the same as the prior ARPI. In some embodiments, the prior ARPI therapy is enzalutamide.

In some embodiments, the ARPI is abiraterone. In some embodiments, the abiraterone is administered (a) orally; and/or (b) at the dose of about 1000 mg; and/or (c) once daily; and/or (d) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

In some embodiments, the corticosteroid is prednisone. In some embodiments, the prednisone is administered (a) orally; and/or (b) at a dose of about 5 mg; and/or (c) every 12 hours; and/or (d) twice daily; and/or (e) at a total dose of about 10 mg per day; and/or (f) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

Provided in one aspect is a method of treating mCRPC in a subject in need thereof comprising administering to the subject a besylate salt of an LSD-1 inhibitor compound having the structure:

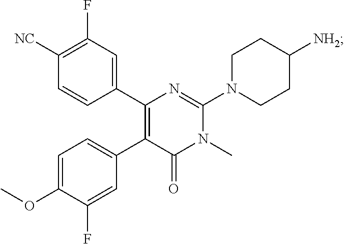

wherein, the LSD-1 inhibitor reverses the castration resistance due to lineage switch.

In some embodiments, the method further comprises the step of administering an ARPI and a corticosteroid, such as prednisone, in combination with the LSD-1 inhibitor. In some embodiments, the subject has failed prior ARPI therapy and the ARPI is either different or the same as the prior ARPI. In some embodiments, the prior ARPI therapy is enzalutamide.

In some embodiments, the ARPI is abiraterone. In some embodiments, the abiraterone is administered (a) orally; and/or (b) at the dose of about 1000 mg; and/or (c) once daily; and/or (d) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

In some embodiments, the prednisone is administered (a) orally; and/or (b) at a dose of about 5 mg; and/or (c) every 12 hours; and/or (d) twice daily; and/or (e) at a total dose of about 10 mg per day; (f) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

Provided in another aspect is a method of treating mCRPC in a subject who has failed prior therapy with enzalutimide comprising: a first step of administering to the subject a besylate salt of an LSD-1 inhibitor compound having the structure:

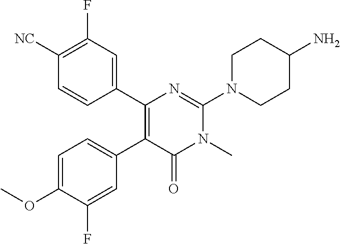 [SMILES: COc1ccc(-c2c(-c3ccc(C#N)c(F)c3)nc(N3CCC(N)CC3)n(C)c2=O)cc1F]

in a 28-day cycle:
(a) orally; and/or
(b) at the dose of about 60 mg; and/or
(c) once a week; and/or
(d) on Days 1, 8, 15 and 22 of a 28 day cycle,

wherein the LSD-1 inhibitor reverses the castration resistance due to lineage switch,

thereafter a second step of concomitantly administering the LSD-1 inhibitor, abiraterone, and prednisone, wherein the LSD-1 inhibitor is administered in a 28-day cycle:
(e) orally; and/or
(f) at the dose of about 60 mg, 40 mg, or 20 mg; and/or
(g) once a week; and/or
(h) on Days 1, 8, 15 and 22 of a 28 day cycle,
   wherein the abiraterone is administered:
(i) orally; and/or
(j) at the dose of about 1000 mg; and/or
(k) once daily; and/or
(l) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle, and
   wherein the prednisone is administered:
(m) orally; and/or
(n) at a dose of about 5 mg; and/or
(o) every 12 hours; and/or
(p) twice daily; and/or
(q) at a total dose of about 10 mg per day;
(r) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

In some embodiments, the tumor has reduced in size, or prostate specific antigen (PSA) levels have decreased compared to baseline.

Both the foregoing summary and the following description of the drawings and detailed description are exemplary and explanatory. They are intended to provide further details of the invention, but are not to be construed as limiting. Other objects, advantages, and novel features will be readily apparent to those skilled in the art from the following detailed description of the invention.

### BRIEF DESCRIPTION OF THE DRAWINGS

FIGS. 1A and 1B show PC3 cells treated with indicated concentrations of Compound A for 3 days. Proliferation was measured both at Day 0 and at Day 3, and percent of growth was determined using a CellTiter Glo luminescent assay.

FIGS. 2A and 2B show LNCaP-AR CRISPRi TP53/RB1 cells treated with indicated concentrations of Compound A and enzalutamide 10 uM on Day 0 and on Day 3 for a total of 6 days. Proliferation was measured both at Day 0 and at Day 6, and percent of growth was determined using a CellTiter Glo luminescent assay.

FIGS. 3A and 3B show TKO cells treated with indicated concentrations of Compound A and enzalutamide 10 uM on Day 0 and on Day 3 for a total of 6 days. Proliferation was measured both at Day 0 and at Day 6, and percent of growth was determined using a CellTiter Glo luminescent assay.

FIGS. 3C and 3D show TKO cells treated with indicated concentrations of Compound A and enzalutamide 10 uM on Day 0 and on Day 6 for a total of 14 days. Proliferation was measured both at Day 0 and at Day 14, and percent of growth was determined using a CellTiter Glo luminescent assay.

FIGS. 4A and 4B show DKO cells treated with indicated concentrations of Compound A and enzalutamide 10 uM on Day 0 and on Day 6 for a total of 14 days. Proliferation was measured both at Day 0 and at Day 14, and percent of growth was determined using a CellTiter Glo luminescent assay.

FIGS. 5A and 5B show TKO and DKO cells treated with indicated concentrations of Compound A and enzalutamide (10 uM) for 8 days. Krt8 gene expression was determined by qPCR.

FIGS. 6A and 6B show LNCaP-AR cells treated with indicated concentrations of Compound A and enzalutamide for 3 days. Proliferation was measured both at Day 0 and at Day 3, and percent of growth was determined using a CellTiter Glo luminescent assay.

FIGS. 7A and 7B show VCaP cells treated with indicated concentrations of Compound A and Enzalutamide 10 uM for 3 days. Proliferation was measured both at Day 0 and at Day 3, and percent of growth was determined using a CellTiter Glo luminescent assay.

FIGS. 8A and 8B show LNCaP cells treated with indicated concentrations of Compound A and enzalutamide 10 uM for 6 days. Proliferation was measured both at Day 0 and at Day 6, and percent of growth was determined using a CellTiter Glo luminescent assay.

FIG. 9 shows the overall study design of Example 2.

### DETAILED DESCRIPTION

### I. Overview

The present invention is directed to methods of treating prostate cancer and/or related symptoms. The methods comprise administering a lysine specific demethylase-1 (LSD-1) inhibitor, or a pharmaceutically acceptable salt thereof to a subject in need thereof, wherein the LSD-1 inhibitor resensitizes the prostate cancer cells to an androgen receptor pathway inhibitor (ARPI). In some instances, the subject has failed prior ARPI therapy. In some embodiments, the method comprises administering to a subject in need thereof an LSD-1 inhibitor, or a pharmaceutically acceptable salt thereof, with an ARPI, or a pharmaceutically acceptable salt thereof.

This disclosure recognizes that in the embodiments described herein, the method enhances the therapeutic effects of the ARPI by resensitizing prostate cancer cells to the ARPI, the LSD-1 inhibitor blocks and/or reverses lineage transformation of the prostate cancer cells, and/or the blockage or reversal results in extending the effectiveness of the ARPI, thereby positively impacting the prostate cancer treatment.

As detailed in Example 1, an LSD-1 inhibitor such as Compound A can be used in combination with at least one ARPI to treat prostate carcinoma cells and to extend the durability of clinically beneficial antiandrogen therapy in CRPC and NEPC cells. The model ARPI used in Example 1 is enzalutamide (ENZA). In particular, the data in Example 1 demonstrates that the LSD-1 inhibitor Compound A can re-sensitize neuroendocrine and/or castration resistant prostate cancer cells to treatment with an ARPI. In addition, LSD-1 inhibitor Compound A was found to enhance ARPI response in prostate carcinoma cells.

Prostate cancer is the second most common malignancy in men in the US. Current standard of care consists of androgen depletion therapy (ADT) but after a rapid remission cancer cells eventually acquire resistance and castration resistant prostate cancer (CRPC) progresses. CRPC is treated with next generation ARPIs including but not limited to enzalutamide. In most cases, after an initial response, disease recurs as androgen-dependent CRPC. A subset of patients develops instead an androgen receptor (AR)-independent disease characterized by a neuroendocrine (NE) phenotype after ARPI treatment. Mechanisms leading to drug resistance and to the development of a NE phenotype are still not completely understood but recent studies suggest that cellular plasticity and reacquisition of stem-cell like properties may lead to resistance.

Various cell and animal models were used to evaluate the effectiveness of anti-cancer treatment utilizing Compound A alone, an ARPI alone, and a combination of Compound A+an ARPI. The cell lines utilized included: (i) VCaP cells, which exhibit characteristics of clinical prostate carcinoma including expression of Prostate-Specific Antigen (PSA) and androgen receptor (AR); (ii) Prostate cancer cells PC3, which are a widely accepted NEPC cell model; (iii) LNCaP-AR cells depleted for Tumor Protein 53 (TP53) and Retinoblastoma protein 1 (RB1); (iv) DKO cells (double knock out) and TKO cells (triple knock out), from genetically engineered mouse models deleted for Phosphatase and Tensin Homolog (Pten), Rb 1, and Trp53, which are used to study NEPC phenotype.

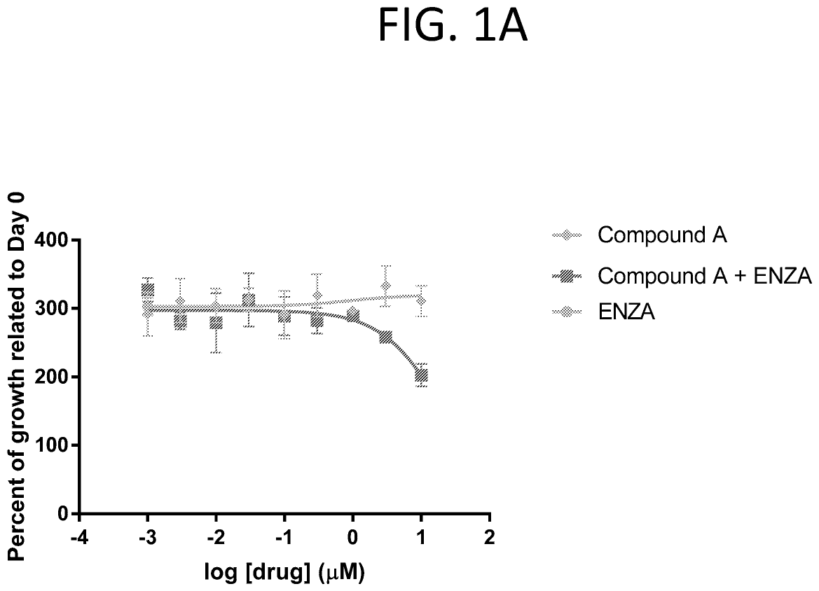

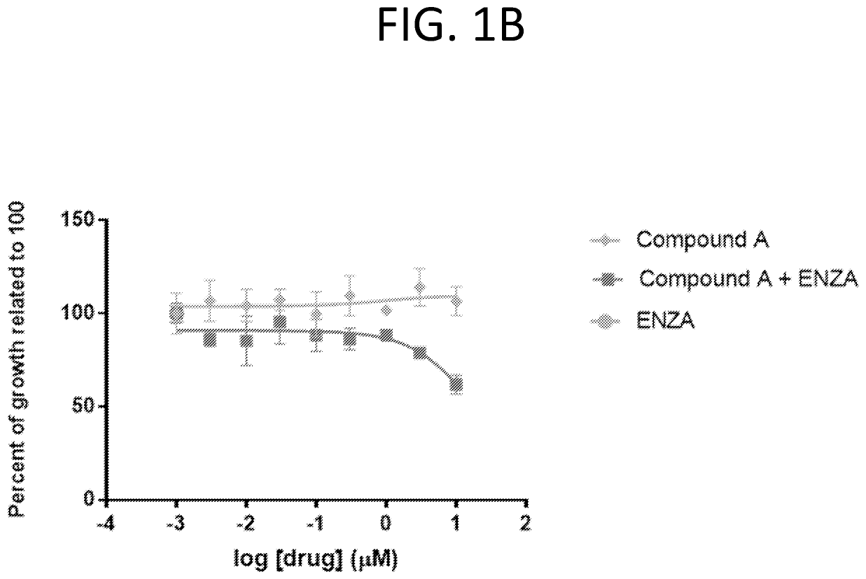

LSD-1 inhibitor+ARPI shows significant inhibition of PC3 cells: Prostate cancer cells PC3 are a widely accepted human NE prostate cancer (NEPC) cell model. To test if an LSD-1 inhibitor may affect cell growth of NEPC, PC3 cells were treated with Compound A and a model ARPI, ENZA. These cells as it has been previously described, and as shown in FIGS. 1A and 1B, are resistant to ENZA. Further, Compound A as a single agent does not affect cell proliferation of PC3 cells. Surprisingly, co-treatment with Compound A and Enzalutamide led to 47% of growth inhibition of PC3 cells (FIGS. 1A and 1B).

Co-treatment with an LSD-1 inhibitor can re-sensitize NEPC to ARPI treatment: Comprehensive next-generation sequencing studies comparing prostate adenocarcinoma with NEPC have identified key genetic alterations. Proto-oncogene MYCN amplification was found in 40% of NEPC samples, RB1 lost in 70-90% of cases, and TP53 lost in 56-67% of cases. Moreover, RB1 lost and TP53 mutation or deletion were found together in 50% of NEPC tumor. Recently a study reported that depletion of TP53 and RB1 in LNCaP cells overexpressing AR promoted lineage plasticity increasing expression of basal and neuroendocrine markers and reducing expression of luminal markers. Moreover, silencing of TP53 and RB1 was sufficient to confer resistance to the antiandrogen-sensitive LNCaP-AR prostate cells.

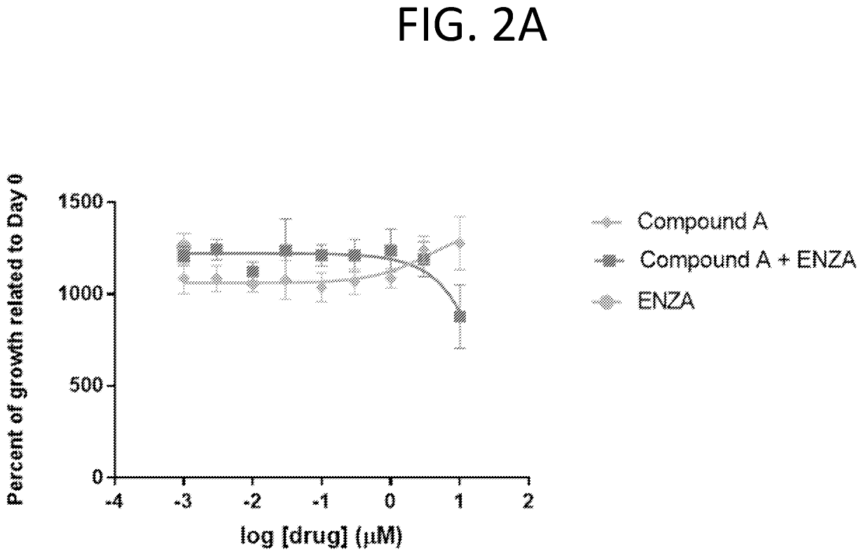

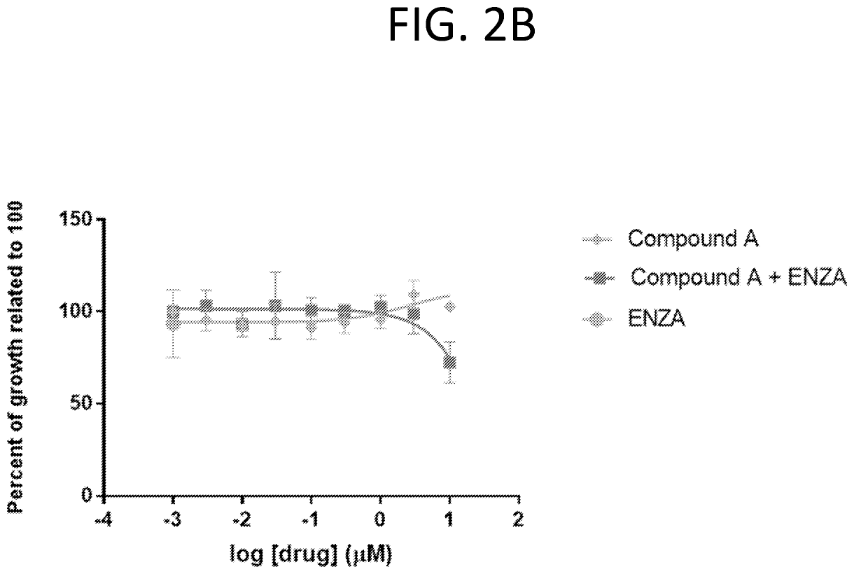

To further corroborate the capability of LSD-1 inhibitor to re-sensitize NEPC to ENZA, LNCaP_AR depleted for TP53 and RB1 using CRISPR interference (CRISPRi) technique (LNCaP_AR guideTP53/RB1) cells were treated with Compound A and ENZA as single agents and in combination. The results showed that Compound A or ENZA alone did not affect proliferation of these cells (FIGS. 2A and 2B). However, surprisingly co-treatment with Compound A and ENZA showed a 27% decrease in cell proliferation (FIGS. 2A and 2B).

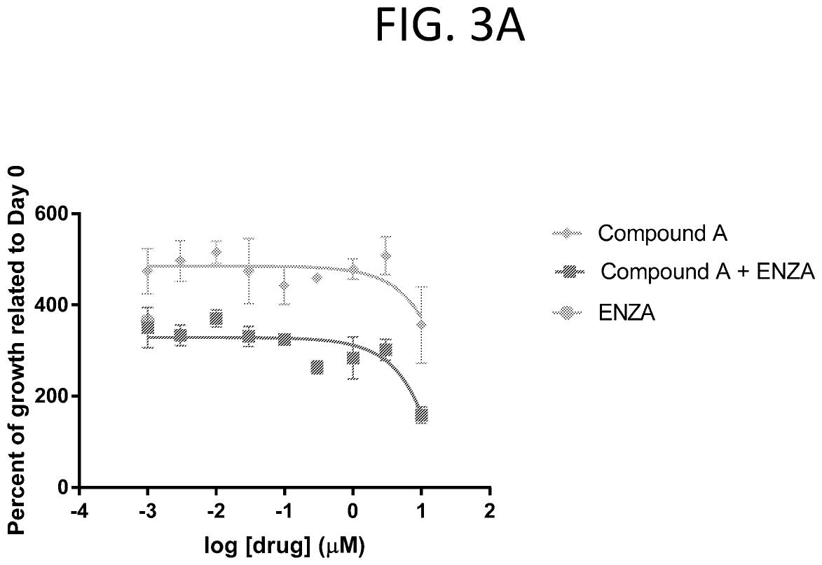

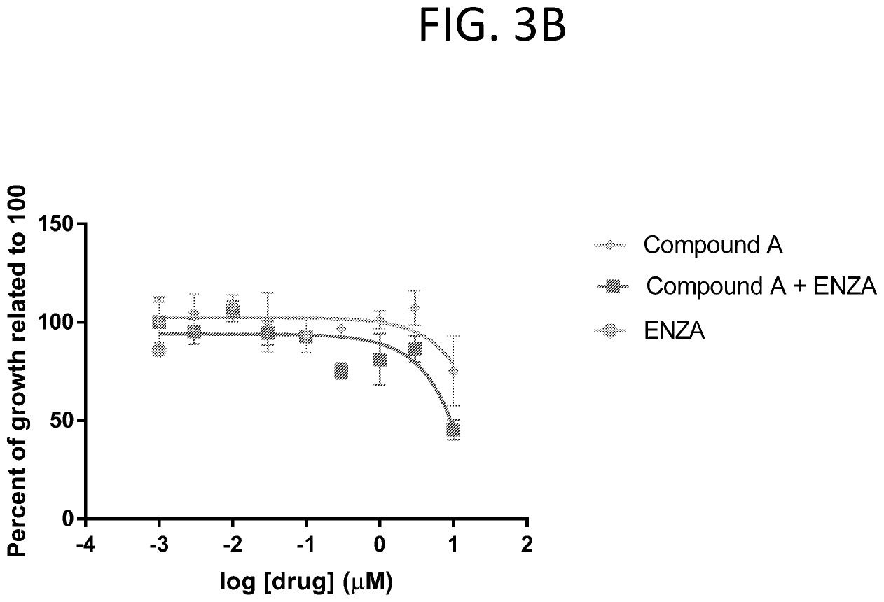

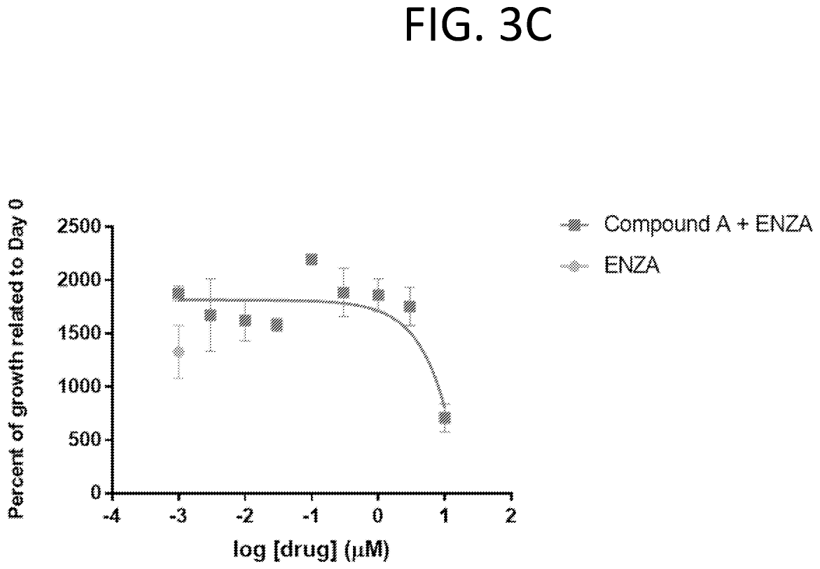

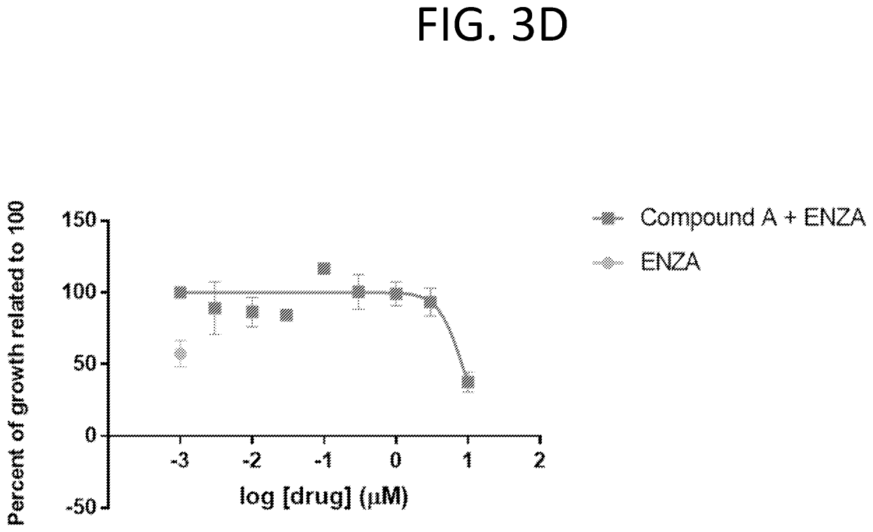

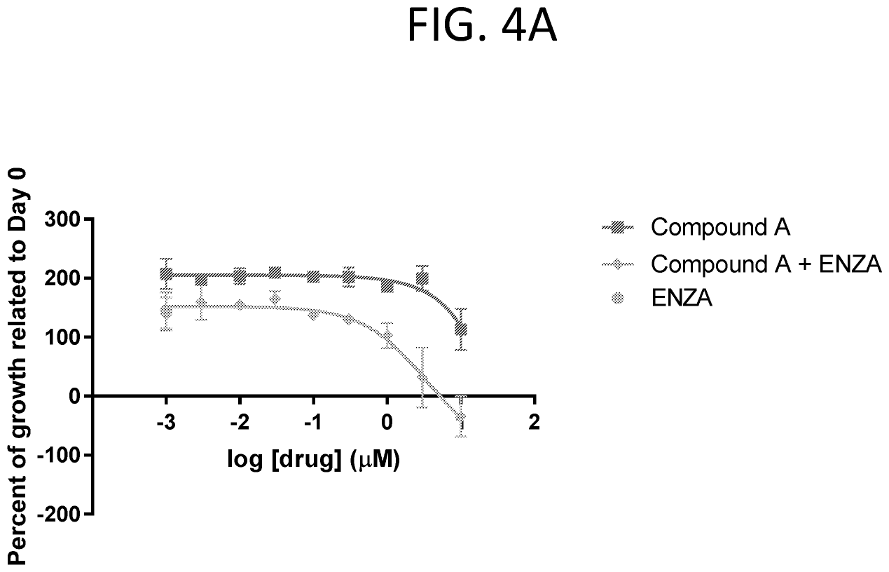

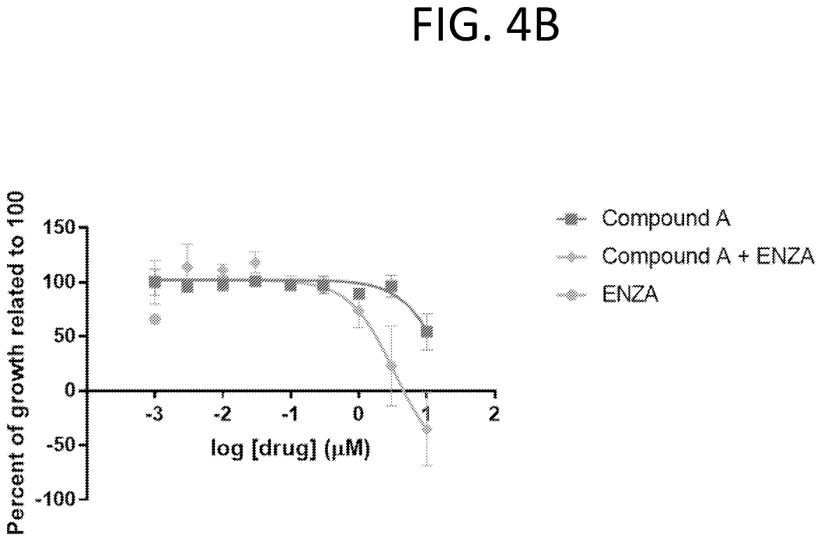

Gene expression profiles have shown that tumors derived from mice deleted for Phosphatase and tensin homolog (Pten), Rb 1, and Trp53 resemble human prostate cancer neuroendocrine variants. Genetically engineered mouse models deleted for Pten, and Rb 1 (double knock out DKO mice), or deleted for Pten, Rb 1 and Trp53 (triple knock out TKO mice) have been used to study NEPC. To test if an LSD1 inhibitor can affect proliferation of these mouse-derived NEPC cells, DKO and TKO cells were treated with Compound A as a single agent and in combination with ENZA. Compound A alone showed a mild effect on both TKO and DKO cell proliferation (FIGS. 3A, 3B, 4A, and 4B). However, when Compound A and the model ARPI ENZA are combined, a greater proliferation decrease is observed. Specifically, in TKO cells a 55% of reduction in cell proliferation was achieved 6 days after treatment, and 65% was observed after 14 days of treatment (FIGS. 3A, 3B, 3C, and 3D). In DKO cells cell growth arrest was achieved with >3 uM of Compound A and higher concentration of Compound A induced cell death (FIGS. 4A and 4B).

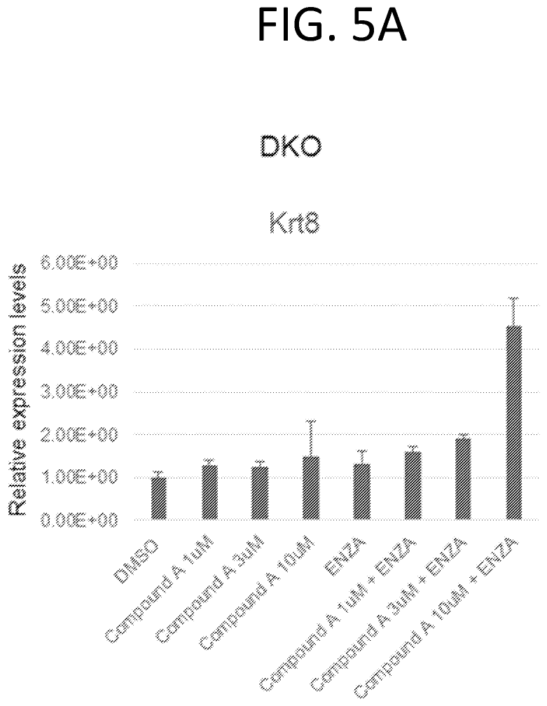

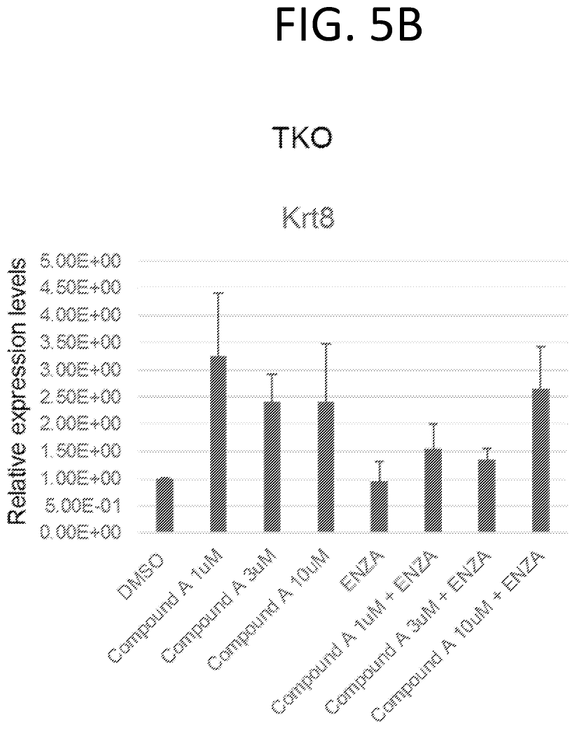

To further confirm that LSD1 inhibition can have a role in NEPC plasticity reversing NE phenotype into an AR-driven epithelial state, DKO and TKO cells were treated with Compound A alone and in combination with ENZA. Compound A as single agent induced the expression of a luminal lineage marker Krt8 in DKO cells, and when combined with ENZA treatment the induction of Keratin8 (Krt8) expression is even more pronounced (1.5-fold and 4.5-fold respectively) (FIGS. 5A and 5B). Further, TKO cells express higher levels of neuroendocrine markers and have been shown to be more resistant to ADT. In TKO cells, Compound A as single agent was able to induce the expression of Krt8 even at lower concentration ≥1 uM (FIGS. 5A and 5B).

These results show that LSD-1 inhibitors such as Compound A are able to re-sensitize NEPC to ARPI treatment by changing lineage transformation.

### LSD-1 Inhibitor Compound a Re-Sensitizes Castration Resistant Prostate Cancer Cells to ARPI Treatment.

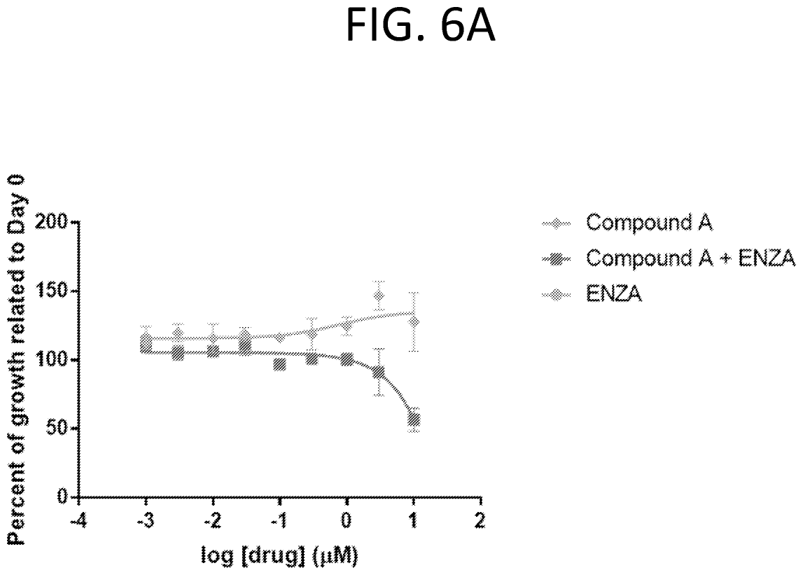

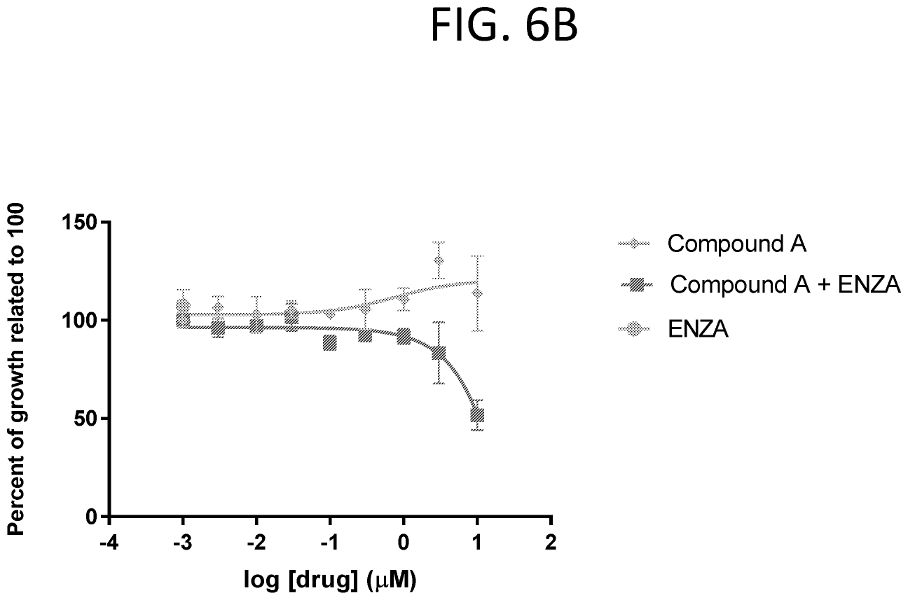

The current standard of care for CRPC consists of ARPI treatment. Overexpression of AR has been previously used as a model of AR-dependent CRPC. To test if LSD-1 inhibition may re-sensitize AR-dependent CRPC cells which have acquired resistance to an ARPI such as ENZA, LNCaP_AR cells were treated with Compound A and ENZA. Compound A as a single agent was found to have no effect on cell proliferation; however, when coupled with ENZA a 50% reduction in cell growth was observed (FIGS. 6A and 6B).

Similar to what has been described in NEPC cells, these data show that an LSD-1 inhibitor such as Compound A can re-sensitize CRPC cells resistant to an ARPI, such as enzalutamide.

### LSD-1 Inhibitor Compound a Enhances Enzalutamide Response in Prostate Carcinoma Cells

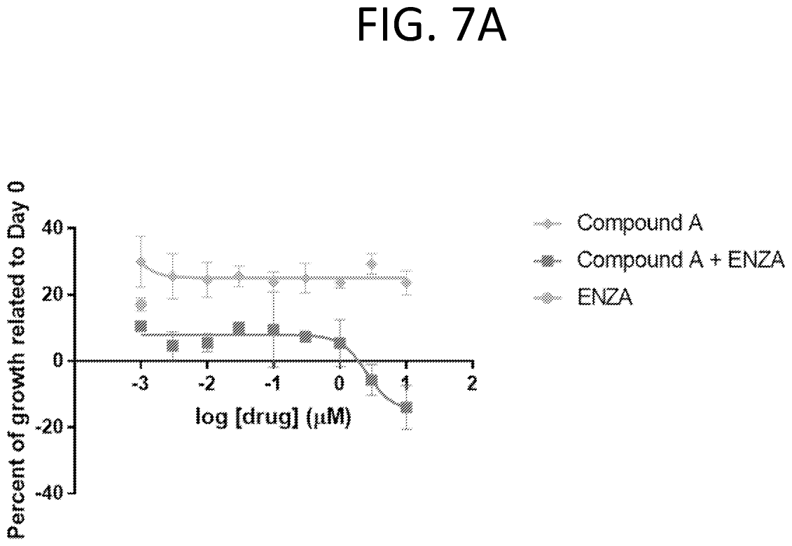

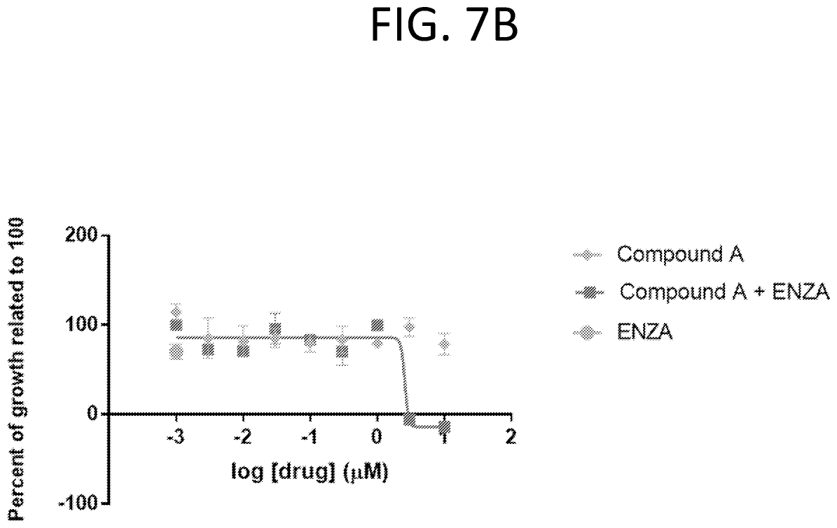

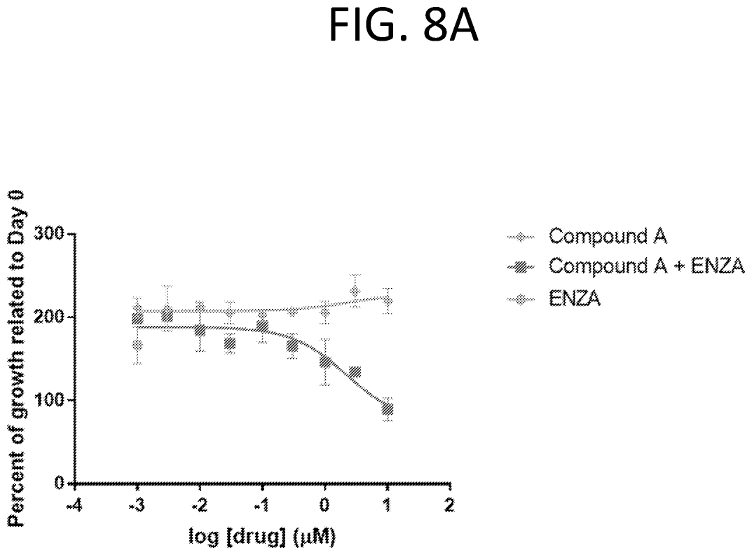

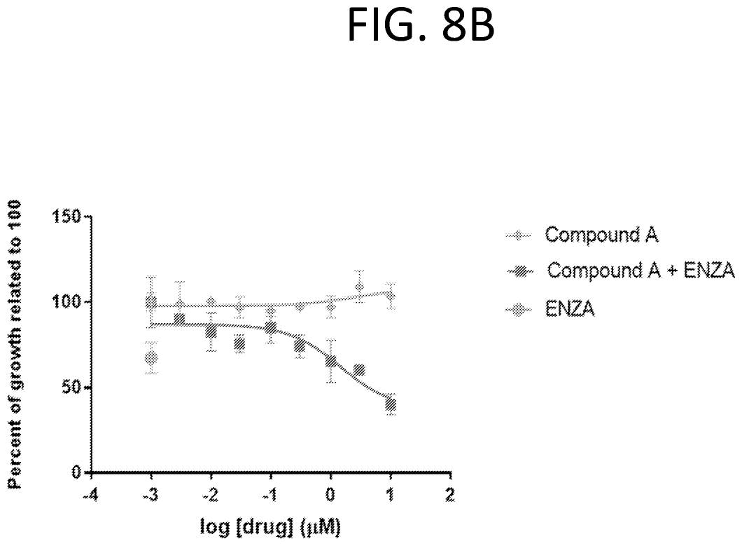

ARPIs such as enzalutamide are currently used as a standard of care in prostate cancer. To test if LSD-1 inhibition may increase the sensitivity to enzalutamide therapy, two well characterized prostate cancer cell models, VCaP and LNCaP, were treated with Compound A alone and in combination with enzalutamide. The results show that Compound A as a single agent did not affect proliferation of either VCaP or LNCaP cells (FIGS. 7A, 7B, 8A, and 8B). However, co-treatment with an ARPI such as ENZA was found to potentiate the effect of ENZA in both of the two cell lines leading to cell death in VCaP cells, and causing 60% reduction in cell proliferation in LNCaP cells (FIGS. 7A, 7B, 8A, and 8B).

### II. LSD-1 Inhibitor

In the embodiments described herein, the LSD-1 inhibitor is a compound having the structure:

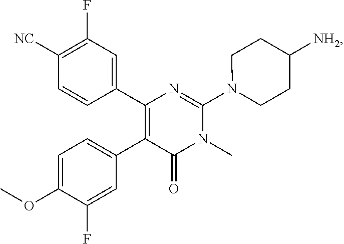 [SMILES: COc1ccc(-c2c(-c3ccc(C#N)c(F)c3)nc(N3CCC(N)CC3)n(C)c2=O)cc1F]

or a pharmaceutically acceptable salt thereof. The chemical name of the above compound is 4-[2-(4-Amino-piperidin-1-yl)-5-(3-fluoro-4-methoxy-phenyl)-6-oxo-1,6-dihydro-pyrimidin-4-yl]-2-fluoro-benzonitrile, with a chemical formula of C₂₃H₂₁F₂N₅O₂, molecular weight of 437.44, and CAS number of 1821307-10-1. 4-[2-(4-amino-piperidin-1-yl)-5-(3-fluoro-4-methoxy-phenyl)-1-methyl-6-oxo-1,6-dihydro-pyrimidin-4-yl]-2-fluoro-benzonitrile is described in U.S. patent application Ser. No. 14/701,304 (U.S. Pat. No. 9,255,097).

### III. Androgen Receptor Pathway Inhibitor (ARPI)

Androgen receptor (AR) is a member of the steroid and nuclear receptor superfamily. Among this large family of proteins, only five vertebrate steroid receptors are known and include the androgen receptor, estrogen receptor, progesterone receptor, glucocorticoid receptor, and mineralocorticoid receptor. AR is a soluble protein that functions as an intracellular transcription factor. AR function is regulated by the binding of androgens, which initiates sequential conformational changes of the receptor that affect receptor-protein interactions and receptor-DNA interactions.

Examples of ARPIs include, but are not limited to, abiraterone, orteronel, bicalutamide, nilutamide, flutamide, hydroxyflutamide, enzalutamide (also known as MDV3100; CAS No: 915087-33-1), apalutamide, darolatamide, galeterone, triptophenolide, dehydroepiandrosterone (DHEA), cyproterone Acetate, spironolactone, RU58841, EPI-001, Andarine, ARN-509 (CAS No. 956104-40-8), RD162 (CAS No. 915087-27-3) and any pharmaceutically acceptable salt thereof.

In any of the embodiments described herein, the ARPI can be enzalutamide or a pharmaceutically acceptable salt thereof. The chemical name for enzalutamide is 4-{3-[4-cyano-3-(trifluoromethyl)phenyl]-5,5-dimethyl-4-oxo-2-sulfanylideneimidazolidin-1-yl}-2-fluoro-N-methylbenzamide. The structure of enzalutamide is shown below:

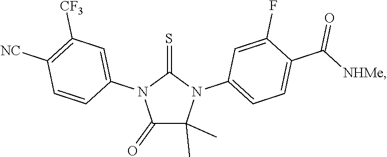 [SMILES: CNC(=O)c1ccc(N2C(=S)N(c3ccc(C#N)c(C(F)(F)F)c3)C(=O)C2(C)C)cc1F]

and has the molecular weight of 464.44 and molecular formula of C₂₁H₁₆F₄N₄O₂S.

In any of the embodiments described herein, the ARPI can be abiraterone. In any of the embodiments described herein, the ARPI can be abiraterone or a pharmaceutically acceptable salt thereof, such as the acetate. The chemical name for abiraterone is (3β)-17-(pyridin-3-yl)androsta-5,16-dien-3-ol. The structure of abiraterone is shown below:

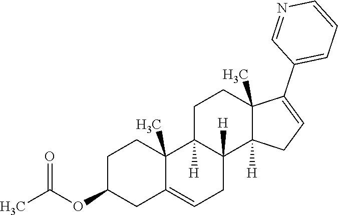

Abiraterone acetate has the molecular weight of 391.55 and molecular formula of C₂₆H₃₃NO₂.

In some embodiments, the ARPI is administered with a corticosteroid. Examples of suitable corticosteroids include, but are not limited to, prednisone, methylprednisolone, hydrocortisone and dexamethasone. In some embodiments, the corticosteroid is prednisone.

### IV. Methods of Treatment

The present application provides methods for the treatment of prostate cancer using a LSD-1 inhibitor in combination with at least one ARPI, or a pharmaceutically acceptable salt thereof. In some embodiments, the treatment of prostate cancer is with 4-[2-(4-Amino-piperidin-1-yl)-5-(3-fluoro-4-methoxy-phenyl)-6-oxo-1,6-dihydro-pyrimidin-4-yl]-2-fluoro-benzonitrile co-administered or sequentially administered with at least one ARPI or a pharmaceutically acceptable salt thereof. In some embodiments, the treatment of prostate cancer is with an LSD-1 inhibitor and the ARPI enzalutamide, or a pharmaceutically acceptable salt thereof. In some embodiments, the treatment of prostate cancer is with 4-[2-(4-Amino-piperidin-1-yl)-5-(3-fluoro-4-methoxy-phenyl)-6-oxo-1,6-dihydro-pyrimidin-4-yl]-2-fluoro-benzonitrile and enzalutamide, or a pharmaceutically acceptable salt thereof. In some embodiments, the treatment of prostate cancer is with an LSD-1 inhibitor and the ARPI abiraterone, or a pharmaceutically acceptable salt thereof. In some embodiments, the treatment of prostate cancer is with 4-[2-(4-Amino-piperidin-1-yl)-5-(3-fluoro-4-methoxy-phenyl)-6-oxo-1,6-dihydro-pyrimidin-4-yl]-2-fluoro-benzonitrile and abiraterone, or a pharmaceutically acceptable salt thereof. In some embodiments, the treatment of an ARPI is in combination with a corticosteroid, such as prednisone.

In any of the embodiments described herein, a therapeutically effective amount of the LSD-1 inhibitor and/or the ARPI is used. In some embodiments, the LSD-1 inhibitor, or a pharmaceutically acceptable salt thereof, enhances the therapeutic benefit of the ARPI. In some embodiments, the LSD-1 inhibitor, or a pharmaceutically acceptable salt thereof, blocks and/or reverses lineage transformation of the prostate cancer cells. In some embodiments, the blockage or reversal results in extending the effectiveness of the ARPI, thereby positively impacting the prostate cancer treatment. In some embodiments, the LSD-1 inhibitor, or a pharmaceutically acceptable salt thereof, is in an amount to substantially induce the cell cycle arrest of the prostate cancer.

In some embodiments, the method of treatment comprises two phases: a first phase where the LSD-1 inhibitor is administered first as a monotherapy in the first 28-day cycle; and a second phase where the LSD-1 inhibitor is administered in combination with at least one ARPI (with or without a corticosteroid) in the second and subsequent 28-day cycles. If the dose of the LSD-1 inhibitor is not tolerated when administered in combination with the at least one ARPI, then the dose of the LSD-1 inhibitor is adjusted to a lower dose (e.g. from 60 mg to 40 mg or 20 mg per day). For instance, if a dose of about 60 mg of LSD-1 inhibitor is not tolerated in combination with the ARPI, such as abiraterone, then the dose of the LSD-1 inhibitor is decreased to a dose of about 20 mg or about 40 mg.

Provided in one aspect is a method of treating mCRPC in a subject in need thereof comprising administering to the subject a besylate salt of an LSD-1 inhibitor compound having the structure:

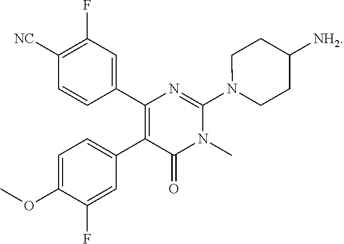 [SMILES: COc1ccc(-c2c(-c3ccc(C#N)c(F)c3)nc(N3CCC(N)CC3)n(C)c2=O)cc1F]

In some embodiments, in a first 28-day cycle, the LSD-1 inhibitor is administered orally. In some embodiments, in a first 28-day cycle, the LSD-1 inhibitor is administered at the dose of from about 20 mg to about 60 mg, including about 20 mg, 40 mg, and 60 mg. In some embodiments, in a first 28-day cycle, the LSD-1 inhibitor is administered at the dose of about 60 mg. In some embodiments, in a first 28-day cycle, the LSD-1 inhibitor is administered once a week. In some embodiments, in a first 28-day cycle, the LSD-1 inhibitor is administered on Days 1, 8, 15 and 22 of a 28 day cycle.

In some embodiments, the subject has failed prior androgen receptor pathway inhibitor therapy. As used herein, failure to prior androgen receptor pathway inhibitor therapy may be defined as tumor progression (increase in size), no reduction in tumor size, or/and prostate-specific antigen (PSA) progression compared to baseline. Tumor progression may be assessed by radiographic progression of soft tissue disease by Response Evaluation Criteria In Solid Tumor (RECIST), Version 1.1 or bone metastasis with 2 or more documented new bone lesions on a bone scan with or without PSA progression. PSA progression is defined by a minimum of 3 rising PSA levels with an interval of ≥1 week between each determination. The PSA value at screening must be ≥1 μg/L (1 ng/mL) if PSA is the only indication of progression; subjects on systemic glucorticoids for control of symptoms must have documented PSA progression by Prostate Cancer Clinical Trials Working Group (PCWG3) criteria (Scher et al., J Clin Oncol., 34:1402-1418 (2016)) while on systemic glucocorticoids prior to commencing monotherapy treatment with a LSD-1 inhibitor. In some embodiments, PSA progression is defined as an increase in PSA greater than about 25% and >about 2 ng/ml above nadir, confirmed by progression at 2 timepoints at least 3 weeks apart.

In some embodiments, the prior ARPI therapy is enzalutamide.

In some embodiments, the method further comprises the step of administering an androgen receptor pathway inhibitor and a corticosteroid, such as prednisone, in combination with the LSD-1 inhibitor. In some embodiments, the subject has failed prior androgen receptor pathway inhibitor therapy and the androgen receptor pathway inhibitor is either different or the same as the prior androgen receptor pathway inhibitor. In some embodiments, the prior androgen receptor pathway inhibitor therapy is enzalutamide.

In some embodiments, the androgen receptor pathway inhibitor is abiraterone. In some embodiments, the abiraterone is administered orally. In some embodiments, the abiraterone is administered at a dose of from about 250 to about 2000 mg or from about 250 to about 1000 mg, including about 250 mg, about 500 mg, about 750 mg, about 1000 mg, about 1250 mg, about 1500 mg, about 1750 mg, about 2000 mg. In some embodiments, the abiraterone is administered at a dose of about 1000 mg. In some embodiments, the abiraterone is administered once or twice daily. In some embodiments, the abiraterone is administered once daily. In some embodiments, the abiraterone is administered at a dose of about 1000 mg once a day. In some embodiments, the abiraterone is administered on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

In some embodiments, the corticosteroid is prednisone. In some embodiments, the prednisone is administered orally. In some embodiments, the prednisone is administered at a dose of about 2.5 mg, about 5 mg, or about 10 mg. In some embodiments, the prednisone is administered at a dose of about 5 mg. In some embodiments, the prednisone is administered every 12 hours. In some embodiments, the prednisone is administered at a dose of about 5 mg at every 12 hours. In some embodiments, the prednisone is administered once or twice daily. In some embodiments, the prednisone is administered twice daily. In some embodiments, the prednisone is administered at a total dose of about 2.5 mg, about 5 mg, or about 10 mg per day. In some embodiments, the prednisone is administered at a total dose of about 10 mg per day. In some embodiments, the prednisone is administered on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

Also provided in one aspect is a method of treating metastatic castration-resistant prostate cancer (mCRPC) in a subject in need thereof comprising administering to the subject a besylate salt of an LSD-1 inhibitor compound having the structure:

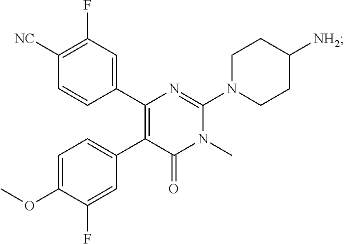

wherein, the LSD-1 inhibitor reverses the castration resistance due to lineage switch.

Assessing the reversal of castration resistance due to lineage switch may be determined by any one of the following as described in Example 2 or any combination thereof: (i) imaging data of 18-fluoro-deoxyglucose (FDG/FDHT) uptake; (ii) biomarker data on biopsies, on circulating tumor DNA (ctDNA), circulating tumor cells (CTC), neuroendocrine peptides PD (NEPD), and prostate-specific antigen (PSA); and (iii) clinical response. See, Berger, Nat Rev Clin Oncol., 15(6): 353-365 (2018).

The reversal of castration resistance due to lineage switch may be defined one or more of the following: (a) at least a 30% increase from baseline FDHT-PET as described in Example 2; and/or (b) a change of from about 10% to about 90% in circulating tumor cells (CTC); and/or (c) a change of from about 10% to about 90% in circulating tumor DNA (ctDNA) analyses; and/or (d) a change of from about 10% to about 90% in serum Neuro Endocrine Peptide PD (NEPD) markers, which include, but are not limited to, to Pro-GRP, CgA, SYP and NSE; and/or (e) a change of from about 10% to about 90% in androgen receptor levels from tumor biopsies as determined by assessing androgen receptor DNA and RNA markers.

In some embodiments, the reversal of castration resistance due to lineage switch is defined by at least a 10% increase, at least a 15% increase, at least a 20% increase, at least a 25% increase from baseline FDHT-PET as described in Example 2. In some embodiments, the reversal of castration resistance due to lineage switch is defined by at least a 30% increase from baseline FDHT-PET as described in Example 2. An exemplary protocol for using FDHT-PET imaging is described in Kelloff, Clin Cancer Res. 2005 Apr. 15; 11(8):2785-808 and Wahl, J. Nucl. Med., 2009 May; 50 Suppl 1:122S-50S.

In some embodiments, the reversal of castration resistance due to lineage switch is defined by a change of from about 10% to about 90% in circulating tumor cells (CTC). In some embodiments, the reversal of castration resistance due to lineage switch is defined by a change of about 10%, about 20%, about 30%, about 40%, about 50%, about 60%, about 70%, about 80%, or about 90% in circulating tumor cells (CTC). An exemplary protocol for using circulating tumor cells is described in Krebs, Ther Adv Med Oncol. 2010; 2(6):351-365.

In some embodiments, the reversal of castration resistance due to lineage switch is defined by a change of from about 10% to about 90% in circulating tumor DNA (ctDNA) analyses. In some embodiments, the reversal of castration resistance due to lineage switch is defined by a change of about 10%, about 20%, about 30%, about 40%, about 50%, about 60%, about 70%, about 80%, or about 90% in circulating tumor DNA (ctDNA) analyses. An exemplary protocol for using circulating tumor DNA analyses is described in Donaldson, Annu Rev Med. 2018 Jan. 29; 69:223-234.

In some embodiments, the reversal of castration resistance due to lineage switch is defined by a change of from about 10% to about 90% in serum Neuro Endocrine Peptide PD (NEPD) markers. In some embodiments, the reversal of castration resistance due to lineage switch is defined by a change of about 10%, about 20%, about 30%, about 40%, about 50%, about 60%, about 70%, about 80%, or about 90% in serum Neuro Endocrine Peptide PD (NEPD) markers. Exemplary NEPD markers include, but are not limited to, to Pro-GRP, CgA, SYP and NSE.

In some embodiments, the reversal of castration resistance due to lineage switch is defined by a change of from about 10% to about 90% in androgen receptor levels from tumor biopsies as determined by assessing androgen receptor DNA and RNA markers. In some embodiments, the reversal of castration resistance due to lineage switch is defined by a change of about 10%, about 20%, about 30%, about 40%, about 50%, about 60%, about 70%, about 80%, or about 90% in androgen receptor levels from tumor biopsies as determined by assessing androgen receptor DNA and RNA markers.

In some embodiments, the method further comprises the step of administering an ARPI and a corticosteroid, such as prednisone, in combination with the LSD-1 inhibitor. In some embodiments, the subject has failed prior ARPI therapy and the ARPI is either different or the same as the prior ARPI. In some embodiments, the prior ARPI therapy is enzalutamide.

In some embodiments, the ARPI is abiraterone. In some embodiments, the abiraterone is administered orally. In some embodiments, the abiraterone is administered at the dose of from about 250 to about 2000 mg or from about 250 to about 1000 mg, including about 250 mg, about 500 mg, about 750 mg, about 1000 mg, about 1250 mg, about 1500 mg, about 1750 mg, about 2000 mg. In some embodiments, the abiraterone is administered at the dose of about 1000 mg. In some embodiments, the abiraterone is administered once or twice daily. In some embodiments, the abiraterone is administered once daily. In some embodiments, the abiraterone is administered at the dose of about 1000 mg once a day. In some embodiments, the abiraterone is administered on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

In some embodiments, the prednisone is administered orally. In some embodiments, the prednisone is administered at a dose of about 2.5 mg, about 5 mg, about 10 mg. In some embodiments, the prednisone is administered at a dose of about 5 mg. In some embodiments, the prednisone is administered every 12 hours. In some embodiments, the prednisone is administered at a dose of about 5 mg at every 12 hours. In some embodiments, the prednisone is administered once or twice daily. In some embodiments, the prednisone is administered twice daily. In some embodiments, the prednisone is administered at a total dose of about 2.5 mg, about 5 mg, or about 10 mg per day. In some embodiments, the prednisone is administered at a total dose of about 10 mg per day. In some embodiments, the prednisone is administered on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

Provided in another aspect is a method of treating metastatic castration-resistant prostate cancer (mCRPC) in a subject who has failed prior therapy with enzalutimide comprising: a first step of administering to the subject a besylate salt of an LSD-1 inhibitor compound having the structure:

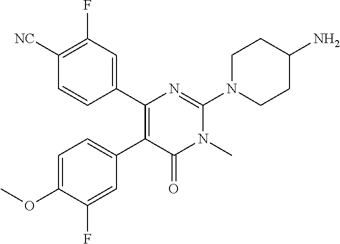 [SMILES: COc1ccc(-c2c(-c3ccc(C#N)c(F)c3)nc(N3CCC(N)CC3)n(C)c2=O)cc1F]

in a 28-day cycle:
(a) orally; and/or
(b) at the dose of about 60 mg; and/or
(c) once a week; and/or
(d) on Days 1, 8, 15 and 22 of a 28 day cycle,

wherein the LSD-1 inhibitor reverses the castration resistance due to lineage switch, thereafter

a second step of concomitantly administering the LSD-1 inhibitor, abiraterone, and prednisone, wherein the LSD-1 inhibitor is administerd in a 28-day cycle:
(e) orally; and/or
(f) at the dose of about 60 mg, about 40 mg, or about 20 mg; and/or
(g) once a week; and/or
(h) on Days 1, 8, 15 and 22 of a 28 day cycle,
   wherein the abiraterone is administered:
(i) orally; and/or
(j) at the dose of about 1000 mg; and/or
(k) once daily; and/or
(l) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle, and
   wherein the prednisone is administered:
(m) orally; and/or
(n) at a dose of about 5 mg; and/or
(o) every 12 hours; and/or
(p) twice daily; and/or
(q) at a total dose of about 10 mg per day;
(r) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

In some embodiments, the failure to prior ARPI therapy is defined as tumor progression (increase in size), no reduction in tumor size, or/and PSA progression compared to baseline as described herein.

In some embodiments, the treatment methods described herein reduces the size of a tumor and/or decreases prostate specific antigen (PSA) levels as compared to baseline.

In one aspect of the methods described herein, the methods result in substantially inducing cell cycle arrest of prostate cancer cell, wherein “substantially” is defined as at least about 50%, at least about 55%, at least about 60%, at least about 65%, at least about 70%, at least about 75%, at least about 80%, at least about 85%, at least about 90%, at least about 95%, or at least about 100% cell cycle arrest of the prostate cancer cell. The percent cell cycle arrest of the prostate cancer cells can be measured using any clinically acceptable technique.

In another aspect, the methods result in completely inducing cell cycle arrest of the prostate cancer. The cell cycle arrest of the prostate cancer cells can be measured using any clinically acceptable technique.

Finally, in another aspect of the methods described herein, the methods result in inducing apoptosis of androgen independent cancer cells. For example, the methods can result in inducing about 20% or more, about 25% or more, about 30% or more, about 35% or more, about 40% or more, about 45% or more, about 50% or more, about 55% or more, about 60% or more, about 65% or more, about 70% or more, about 75% or more, about 80% or more, about 85% or more, about 90% or more, about 95% or more, or about 100% apoptosis of androgen independent cancer cells. Apopotosis of the androgen independent cancer cells can be measured using any clinically acceptable technique.

Provided in one aspect is a method of treating prostate cancer in a subject in need thereof comprising administering to the subject a composition comprising LSD-1 inhibitor, an ARPI, or pharmaceutically acceptable salts thereof.

In any of the embodiments, described herein, the prostate cancer is CPRC, neuroendocrine prostate cancer (NEPC), anti-androgen resistant prostate cancer, enzalutamide resistant prostate cancer, abiraterone resistant prostate cancer, ARPI induced drug resistant prostate cancer, metastatic prostate cancer, or any combination thereof.

In any of the embodiments described herein, the method results in (a) at least about 40% reduction of cancer cell proliferation; (b) from about 40% to about 99% reduction of cancer cell proliferation; and/or (c) in about 20%, about 25%, about 30%, about 35%, about 40%, about 45%, about 50%, about 55%, about 60%, about 65%, about 70%, about 75%, about 80%, about 85%, about 90%, about 95%, or about 99% reduction of cancer cell proliferation. In some embodiments, the method results in at least about 40% reduction of cancer cell proliferation. In some embodiments, the method results in from about 40% to about 99% reduction of cancer cell proliferation. In some embodiments, the method results in about 20%, about 25%, about 30%, about 35%, about 40%, about 45%, about 50%, about 55%, about 60%, about 65%, about 70%, about 75%, about 80%, about 85%, about 90%, about 95%, or about 99% reduction of cancer cell proliferation.

In any of the embodiments described herein, the method results in (a) at least about 40% reduction of tumor size; (b) from about 40% to about 99% reduction of tumor size; and/or (c) about 20%, about 25%, about 30%, about 35%, about 40%, about 45%, about 50%, about 55%, about 60%, about 65%, about 70%, about 75%, about 80%, about 85%, about 90%, about 95%, or about 99% reduction of tumor size. In some embodiments, the method results in at least about 40% reduction of tumor size. In some embodiments, the method results in from about 40% to about 99% reduction of tumor size. In some embodiments, the method results in about 20%, about 25%, about 30%, about 35%, about 40%, about 45%, about 50%, about 55%, about 60%, about 65%, about 70%, about 75%, about 80%, about 85%, about 90%, about 95%, or about 99% reduction of tumor size.

In any of the embodiments described herein, the method provides a greater reduction of cancer cell proliferation and/or tumor size as compared to the administration of the ARPI or the LSD-1 inhibitor as a single agent. In some embodiments, the method provides a greater reduction of cancer cell proliferation as compared to the administration of the ARPI or the LSD-1 inhibitor as a single agent. In some embodiments, the method provides a greater reduction of tumor size as compared to the administration of the ARPI or the LSD-1 inhibitor as a single agent.

In any of the embodiments described herein, the reduction in cancer cell proliferation and/or tumor size can be at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least 95%, or at least about 100% greater than that observed with administration of the ARPI or the LSD-1 inhibitor, or the pharmaceutically acceptable salt thereof, as a single agent.

In any of the embodiments described herein, the reduction in cancer cell proliferation and/or tumor size can be from about 5% to about 100% greater than that observed with administration of the ARPI or the LSD-1 inhibitor, or the pharmaceutically acceptable salt thereof, as a single agent. In any of the embodiments described herein, the reduction in cancer cell proliferation and/or tumor size is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% greater than that observed with administration of the ARPI or the LSD-1 inhibitor, or the pharmaceutically acceptable salt thereof, as a single agent.

In some embodiments, the reduction in cancer cell proliferation is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than that observed with administration of the ARPI or the LSD-1 inhibitor, or the pharmaceutically acceptable salt thereof, as a single agent.

In some embodiments, the reduction in cancer cell proliferation is from about 5% to about 100% greater than that observed with administration of the ARPI or the LSD-1 inhibitor, or the pharmaceutically acceptable salt thereof, as a single agent. In any of the embodiments described herein, the reduction in cancer cell proliferation and/or tumor size is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% greater than that observed with administration of the ARPI or the LSD-1 inhibitor, or the pharmaceutically acceptable salt thereof, as a single agent.

In some embodiments, the reduction in tumor size is at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100% greater than that observed with administration of the ARPI or the LSD-1 inhibitor, or the pharmaceutically acceptable salt thereof, as a single agent.

In some embodiments, the reduction in tumor size is from about 5% to about 100% greater than that observed with administration of the ARPI or the LSD-inhibitor, or the pharmaceutically acceptable salt thereof, as a single agent. In any of the embodiments described herein, the reduction in cancer cell proliferation and/or tumor size is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% greater than that observed with administration of the ARPI or the LSD-1 inhibitor, or the pharmaceutically acceptable salt thereof, as a single agent.

### V. The Response Evaluation Criteria in Solid Tumors (RECIST) Guidelines

As described herein, Response evaluation criteria in solid tumors (RECIST) can be used to measure how a cancer patient responds to treatment. The following information is extracted/summarized from Eisenhauer, 2009, New Response Evaluation Criteria in Solid Tumors: Revised RECIST Guideline (Version 1.1) (Eur J Cancer. 2009 January; 45(2):228-47.)

At screening, tumor lesions/lymph nodes will be categorized as measurable or non-measurable.

Measurable disease: Tumor Lesions can be accurately measured in at least one dimension (longest diameter in the plane of measurement is recorded) with a minimum size of:
10 mm by CT scan (CT scan slice thickness no greater than 5 mm)
10 mm caliper measurement by clinical exam (lesions which cannot be accurately measured with calipers should be recorded as non-measurable)
20 mm by chest X-ray

Malignant Lymph Nodes: To be considered pathologically enlarged and measurable, a lymph node must be ≥15 mm in short axis when assessed by CT scan (CT scan slice thickness recommended to be no greater than 5 mm). At baseline and in follow-up, only the short axis will be measured and followed.

Non-measureable Disease: All other lesions, including small lesions (longest diameter <10 mm or pathological lymph nodes with ≥10 to <15 mm short axis) as well as truly non-measurable lesions. Lesions considered truly non-measurable include leptomeningeal disease, ascites, pleural or pericardial effusion, inflammatory breast disease, lymphangitic involvement of skin or lung, abdominal masses/abdominal organomegaly are identified by physical exam that is not measurable by reproducible imaging techniques.

Tumor Response Evaluation: Target lesions: When more than one measurable tumor lesion is present at baseline then all lesions up to a maximum of five lesions total (and a maximum of 2 lesions per organ) representative of all involved organs can be identified as target lesions and can be recorded and measured at baseline. Target lesions should be selected on the basis of their size (lesions with the longest diameter), be representative of all involved organs, but in addition should be those that lend themselves to reproducible repeated measurements. Note that pathological nodes must meet the measurable criterion of a short axis of ≥15 mm by CT scan and only the short axis of these nodes will contribute to the baseline sum. All other pathological nodes (those with short axis ≥10 mm but <15 mm) are considered non-target lesions. Nodes that have a short axis <10 mm are considered non-pathological and are not recorded or followed. At baseline, the sum of the target lesions (longest diameter of tumor lesions plus short axis of lymph nodes: overall maximum of 5) can be recorded.

After Screening, a value is provided on the eCRF for all identified target lesions for each assessment, even if very small. If extremely small and faint lesions cannot be accurately measured but are deemed to be present, a default value of 5 mm may be used. If lesions are too small to measure and indeed are believed to be absent, a default value of 0 mm may be used.

Non-target lesions: All non-measurable lesions (or sites of disease) plus any measurable lesions over and above those listed as target lesions are considered non-target lesions. Measurements are not required but these lesions can be noted at Screening and should be followed as “present,” “absent,” or “unequivocal progression.”

Response Criteria: Target and non-target lesions can be evaluated for response separately, and then the tumor burden as a whole can be evaluated as the overall response.

Target Lesion Response: Target lesions are assessed as follows:
Complete Response (CR). Disappearance of all target lesions. Any pathological lymph nodes (whether target or non-target) must have reduction in short axis to <10 mm.
Partial Response (PR). At least a 30% decrease in the sum of diameters of target lesions, taking as reference the baseline sum diameters.
Progressive Disease (PD). At least a 20% increase in the sum of diameters of target lesions, taking as reference the smallest sum on study (this includes the Screening sum if that is the smallest on study). In addition to the relative increase of 20%, the sum must also demonstrate an absolute increase of at least 5 mm. (Note: the appearance of one or more new lesions is also considered progression).
Stable Disease (SD). Neither sufficient shrinkage to qualify for PR nor sufficient increase to qualify for PD, taking as reference the smallest sum of diameters while on study.

Non-target Lesion Response: Non-target lesions will be assessed as follows:
Complete Response (CR). Disappearance of all non-target lesions and normalization of tumor marker level. All lymph nodes must be non-pathological in size (<10 mm short axis).
Non-CR/Non-PD. Persistence of one or more non-target lesion(s) and/or maintenance of tumor marker level above the normal limits.
Progressive Disease (PD). Unequivocal progression (see comments below) of existing non-target lesions. (Note: the appearance of one or more new lesions is also considered progression).

When the Subject Also Has Measurable Disease: In this setting, to achieve “unequivocal progression” on the basis of the non-target disease, there must be an overall level of substantial worsening in non-target disease such that, even in presence of SD or PR in target disease, the overall tumor burden has increased sufficiently to merit discontinuation of therapy. A modest “increase” in the size of one or more non-target lesions is usually not sufficient to quality for unequivocal progression status. The designation of overall progression solely on the basis of change in non-target disease in the face of SD or PR of target disease will therefore be extremely rare.

When the Subject Has Only Non-measurable Disease: This circumstance arises in some Phase 3 trials when it is not a criterion of study entry to have measurable disease. The same general concepts apply here as noted above; however, in this instance there is no measurable disease assessment to factor into the interpretation of an increase in non-measurable disease burden. Because worsening in non-target disease cannot be easily quantified (by definition: if all lesions are truly non-measurable) a useful test that can be applied when assessing subjects for unequivocal progression is to consider if the increase in overall disease burden based on the change in non-measurable disease is comparable in magnitude to the increase that would be required to declare PD for measurable disease: i.e., an increase in tumor burden representing an additional 73% increase in “volume” (which is equivalent to a 20% increase diameter in a measurable lesion). Examples include an increase in a pleural effusion from “trace” to “large,” an increase in lymphangitic disease from localized to widespread, or may be described in protocols as “sufficient to require a change in therapy.” If “unequivocal progression” is seen, the subject should be considered to have had overall PD at that point. While it would be ideal to have objective criteria to apply to non-measurable disease, the very nature of that disease makes it impossible to do so: therefore, the increase must be substantial.

Overall Response: Overall response should be assessed according to Table A for subjects with target lesions, and Table B for subjects with only non-target lesions.

TABLE A
Time Point Response: Subjects With Target (± Non-target) Disease

| Target Lesions | Non-target Lesion | New | Overall |
|---|---|---|---|
| Response | Response | Lesions | Response |
| CR | CR | No | CR |
| CR | Non-CR/non-PD | No | PR |
| CR | Not evaluated | No | PR |
| PR | Non-PD or not all evaluated | No | PR |
| SD | Non-PD or not all evaluated | No | SD |
| Not all evaluated | Non-PD | No | NE |
| PD | Any | Yes or No | PD |
| Any | PD | Yes or No | PD |
| Any | Any | Yes | PD |

CR = complete response,
PR = partial response,
SD = stable disease,
PD = progressive disease,
NE = inevaluable.

TABLE B
Time Point Response: Subjects With Non-target Disease Only

| Nontarget Lesions Response | New Lesions | Overall Response |
|---|---|---|
| CR | No | CR |
| Non-CR/non-PD | No | Non-CR/non-PD^{a)} |
| Not all evaluated | No | NE |
| Unequivocal PD | Yes or No | PD |
| Any | Yes | PD |

CR = complete response,
PR = partial response,
SD = stable disease,
PD = progressive disease,
NE = inevaluable.
^{a}Non-CR/non-PD” is preferred over “stable disease” for non-target disease since SD is increasingly used as endpoint for assessment of efficacy in some trials so to assign this category when no lesions can be measured is not advised.

Symptomatic Deterioration: Subjects with a global deterioration of health status requiring discontinuation of treatment without objective evidence of disease progression at that time should be reported as ‘symptomatic deterioration’. Symptomatic deterioration is not a descriptor of an objective response: it is a reason for stopping study therapy. The objective response status of such subjects is to be determined by evaluation of target and non-target disease.

### VI. Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

The term “administering” as used herein includes prescribing for administration as well as actually administering, and includes physically administering by the subject being treated or by another.

As used herein, the term “AR inhibitor”, “AR pathway inhibitor” (ARPI) or “AR antagonist” are used interchangeably herein and refer to an agent that inhibits or reduces at least one activity of the Androgen Receptor (AR). Exemplary AR activities include, but are not limited to, co-activator binding, DNA binding, ligand binding, or nuclear translocation.

As used herein “subject,” “patient,” or “individual” refers to any subject, patient, or individual, and the terms are used interchangeably herein. In this regard, the terms “subject,” “patient,” and “individual” includes mammals, and, in particular humans. When used in conjunction with “in need thereof,” the term “subject,” “patient,” or “individual” intends any subject, patient, or individual having or at risk for a specified symptom or disorder.

As used herein, the phrase “therapeutically effective” or “effective” in context of a “dose” or “amount” means a dose or amount that provides the specific pharmacological effect for which the compound or compounds are being administered. It is emphasized that a therapeutically effective amount will not always be effective in achieving the intended effect in a given subject, even though such dose is deemed to be a therapeutically effective amount by those of skill in the art. For convenience only, exemplary dosages are provided herein. Those skilled in the art can adjust such amounts in accordance with the methods disclosed herein to treat a specific subject suffering from a specified symptom or disorder. The therapeutically effective amount may vary based on the route of administration and dosage form.

The terms “treatment,” “treating,” or any variation thereof includes reducing, ameliorating, or eliminating (i) one or more specified symptoms and/or (ii) one or more symptoms or effects of a specified disorder. The terms “prevention,” “preventing,” or any variation thereof includes reducing, ameliorating, or eliminating the risk of developing (i) one or more specified symptoms and/or (ii) one or more symptoms or effects of a specified disorder.

“Pharmaceutically acceptable salt” includes both acid and base addition salts. A pharmaceutically acceptable salt of any one of the substituted heterocyclic derivative compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts. In some embodiments, the pharmaceutically acceptable salt includes the besylate salt.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogen-phosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitro-benzoates, phthalates, benzenesulfonates (besylates or besilates), toluenesulfonates, phenylacetates, citrates, lactates, malates, tartrates, methanesulfonates, and the like. Also contemplated are salts of amino acids, such as arginates, gluconates, and galacturonates (see, e.g., Berge S. M. et al., Pharmaceutical Salts, J. Pharma. Sci. 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like.

### EXAMPLES

As used in the following examples, Compound A refers to 4-[2-(4-amino-piperidin-1-yl)-5-(3-fluoro-4-methoxy-phenyl)-1-methyl-6-oxo-1,6-dihydro-pyrimidin-4-yl]-2-fluoro-benzonitrile, including the besylate salt.

The below list shows the abbreviations used in the following examples.
ADT Androgen depletion therapy
CRPC Castration resistant prostate cancer
ARPIs Androgen receptor pathway inhibitors
AR Androgen receptor
NE Neuroendocrine
NEPC Neuroendocrine prostate cancer
ENZA Enzalutamide
RB1 Retinoblastoma 1
TP53 Tumor protein 53
CRISPRi CRISPR interference
Pten Phosphatase and tensin homolog
DKO Double knock out
TKO Triple knock out
LSD1 Lysine-specific histone demethylase 1
Krt8 Keratin8

### Example 1

The purpose of this example was to evaluate the effectiveness of a method of treating prostate cancer comprising a combination of an ARPI and an LSD-1 inhibitor compound.

The experiments described herein demonstrate how Compound A can re-sensitize neuroendocrine and/or castration resistant prostate cancer cells to treatment with an ARPI, such as enzalutamide. In addition, Compound A was evaluated to see if it could enhance ARPI response in prostate carcinoma cells.

### Materials and Methods

Cell culture: VCaP, LNCaP, and PC3 cancer cell lines were obtained. VCaP cells were cultured in DMEM media supplemented with 8% FBS, LNCaP cells were cultured in RPMI1640 media supplemented with 10% FBS, and PC3 cells were cultured in F12K media supplemented with 10% FBS. LNCaP_AR, and LNCaP_AR CRISPRi gTP53/RB1 cells were obtained from Charles L. Sawyers lab and were cultured in RPMI1640 supplemented with 10% FBS+2 mM L-Glutamine+1 mM Sodium Pyruvate+10 mM Hepes. DKO and TKO cells were obtained from David W. Goodrich lab and were cultured in DMEM media supplemented with 2.5% charcoal-stripped FBS+5 μg/mL of insulin/transferring/selenium (ITS)+10 μg/mL of bovine pituitary extract (BPE)+10 μg/mL of epidermal growth factor+1 μg/mL of cholera toxin. DKO cells have been derived from intact mice so were cultured also in presence of 1 nM R1881.

Proliferation Assay: Cells were plated in a 384-well format and were allowed to adhere 24 h before treatment. Drugs were titrated 1:3 from 10 μM to 0.1 nM with 9 doses in triplicate. Cell proliferation was measured on the day of treatment (Day 0) and on Day 3 or Day 6 or Day 14 using Cell Titer-Glo reagent (Promega) according to the manufacturer's instructions. Cell growth normalized to the DMSO vehicle control and to Day 0 was fitted by nonlinear regression using GraphPad Prism 7.03 (GraphPad Software, Inc.).

Gene expression analysis: RNA was purified from cells using the RNeasy kit (Qiagen) according to the manufacturer's instructions. Quantitative RTPCR was performed using the TaqMan assay (Thermo Fisher Scientific) on the ABI QuantStudio 7 Flex Real-Time PCR System.

### Results

PC3, VCaP, LNCaP-AR Cell Line Studies PC3 cell model: Prostate cancer cells PC3 are a widely accepted human NE prostate cancer (NEPC) cell model. To test if an LSD-1 inhibitor affects cell growth of NEPC, PC3 cells were treated with Compound A and enzalutamide (ENZA). Compound A as a single agent did not affect cell proliferation of PC3 cells. Surprisingly, it was found that co-treatment with Compound A and enzalutamide led to 47% of growth inhibition of PC3 cells (FIGS. 1A and 1B).

Overexpression of AR has been previously used as a model of AR-dependent CRPC. To test if an LSD-1 inhibition re-sensitizes AR-dependent CRPC cells which have acquired resistance to enzalutamide, LNCaP-AR cells, were first treated with Compound A alone were found to have no effect on cell proliferation; however, when enzalutamide was added after the cells were exposed to Compound A, a 50% reduction in cell growth was observed (FIGS. 6A and 6B).

Well characterized cell lines of human cells commonly used as prostate cancer cell models, VCaP and LNCaP, were treated with Compound A alone and in combination with enzalutamide. Compound A as a single agent was found to have no effect on proliferation of either VCaP or LNCaP cells (FIGS. 7A, 7B, 8A, and 8B). However, treatment with the combination of Compound A and enzalutamide was found to potentiate the effect of enzalutamide in both cell lines leading to cell death in VCaP, and causing 60% reduction in cell proliferation in LNCaP cells (FIGS. 7A, 7B, 8A, and 8B).

Tumor Plasticity: LNCaP-AR depleted for TP53 and RB1 cell model: Drugs targeting the androgen receptor (AR) are initially efficacious, but most tumors eventually become resistant. Particularly, prostate cancer cells escape the effects of ADT through a change in lineage identity. Comprehensive next-generation sequencing studies comparing prostate adenocarcinoma with NEPC have identified key genetic alterations in ADT resistant tumors. Proto-oncogene MYCN amplification was found in 40% of NEPC samples, Retinoblastoma 1 (RB1) lost in 70-90% of cases, and Tumor protein 53 (TP53) lost in 56-67% of cases. Moreover, RB1 lost and TP53 mutation or deletion were found together in 50% of NEPC tumor. Functional loss of the tumor suppressors TP53 and RB1 promoted a shift from AR-dependent luminal epithelial cells to AR-independent basal-like cells. This lineage plasticity is enabled by the loss of TP53 and RB1 functions. Thus, LNCaP-AR cell lines depleted for TP53 and RB1 serve as an additional model for ADT resistant tumor cell types.

To further corroborate the capability of LSD-1 inhibitor to re-sensitize NEPC to enzalutamide, LNCaPAR cells depleted for TP53 and RB1 using CRISPR interference (CRISPRi) technique (LNCaP_AR guideTP53/RB1) were treated with Compound A and enzalutamide as single agents and in combination. The results showed that Compound A alone and enzalutamide alone do not affect proliferation of LNCaPAR cells depleted for TP53 and RB1. (FIGS. 2A and 2B). However, surprisingly it was found that co-treatment with Compound A and enzalutimade showed a 27% decrease in the proliferation rate of LNCaPAR cells depleted for TP53 and RB1. (FIGS. 2A and 2B).

Mouse models: Gene expression profiles have shown that tumors derived from mice deleted for phosphatase and tensin homolog (Pten), Rb 1, and Trp53 resemble human prostate cancer neuroendocrine variants. Genetically engineered mouse models deleted for Pten, and Rb 1 (double knock out DKO mice), or deleted for Pten, Rb 1 and Trp53 (triple knock out TKO mice) have been used to study NEPC. To test if Lysine-specific histone demethylase 1 (LSD-1) inhibitor is affecting proliferation of these mouse-derived NEPC cells, DKO and TKO cells have been treated with Compound A as a single agent and in combination with Enzalutamide.

Compound A alone shows a mild effect on both TKO and DKO cell proliferation (FIGS. 3A, 3B, 4A, and 4B). However, when Compound A and enzalutamid are combined a greater proliferation effect is observed. In TKO 55% of reduction in cell proliferation is achieved 6 days after treatment, and 65% is observed after 14 days of treatment (FIGS. 3A, 3B, 3C, and 3D). In DKO a complete (100%) cell growth arrest is achieved with >3 uM of Compound A and higher concentration of Compound A induces cell death (FIGS. 4A and 4B).

To further confirm that LSD-1 inhibition can have a role in NEPC plasticity reversing NE phenotype into an AR-driven epithelial state, DKO and TKO cells were treated with Compound A alone and in combination with Enzalutamide and expression of luminal lineage marker Krt8 was measured. Compound A as single agent induces the expression of a luminal lineage marker Krt8 in DKO cells, and when Compound A was combined with enzalutamide, the induction of Keratin8 (Krt8) expression is even more pronounced (1.5-fold and 4.5-fold respectively) (FIGS. 5A and 5B). Further, TKO cells express higher levels of neuroendocrine markers and have been shown to be more resistant to ADT. In TKO cells, Compound A as single agent was able to induce the expression of Krt8 even at lower concentration ≥1 uM (FIGS. 5A and 5B).

### Example 2

As used in the following example, Compound A refers to 4-[2-(4-amino-piperidin-1-yl)-5-(3-fluoro-4-methoxy-phenyl)-1-methyl-6-oxo-1,6-dihydro-pyrimidin-4-yl]-2-fluoro-benzonitrile, including the besylate salt.

Study Objectives: The primary objective will be to establish whether Compound A reverses the castration resistance biology, due to lineage switch, in subjects with metastatic castration-resistant prostate cancer (mCRPC) who have failed enzalutamide as last prior therapy followed by a dose finding study of Compound A combined with abiraterone and prednisone.

The secondary objectives are: (1) To assess the safety and tolerability of Compound A as a single agent in mCRPC; (2) To assess the safety and tolerability of Compound A in combination with abiraterone and prednisone and to determine the recommended phase 2 dose (RP2D) of Compound A for the combination with abiraterone and prednisone; (3) To assess the preliminary anti-tumor activity of Compound A in combination with abiraterone and prednisone; and (4) To evaluate prostate-specific antigen (PSA) kinetics during treatment.

The exploratory objectives are: (1) To characterize the pharmacokinetics (PK) profile of Compound A when given in combination with abiraterone and prednisone; (2) To evaluate the pharmacodynamics (PD) effects of Compound A on gene expression in peripheral blood and if available, in tumor samples; (3) To evaluate the PD effects of Compound A on secreted neuropeptide (such as Pro-GRP, CgA, SYP and NSE) in blood; (4) To explore the relationship among Compound A dose, plasma exposure, and selected clinical endpoints (eg, measures of toxicities, preliminary activity, and/or biomarkers); and (5) To explore the relationship between Screening, on-treatment, and/or changes in gene expression in tumor samples (if available), secreted neuropeptides levels in blood, circulating tumor DNA (ctDNA) and circulating tumor cells (CTC) analysis, changes in 18-fluoro-deoxyglucose (FDG/FDHT) uptake and clinical response.

Study endpoints are displayed below in Table 1.

TABLE 1
Study Endpoints

| | | | |
|---|---|---|---|
| Primary | Biological | FDG/FDHT PET | At pre-specified |
| | response | imaging will be | timepoints |
| | | compared with | |
| | | Screening to Cycle 1 | |
| | | (monotherapy period) and from Cycle | |
| | | 1 to Cycle 3 (Cycle 2-3 is combined | |
| | | therapy period) to assess changes in | |
| | | AR expression | |
| Secondary | Safety | AEs and | All AEs will be |
| | | SAEs using | monitored |
| | | the NCI CTCAE | and recorded |
| | | criteria, version 5.0 | from the time the |
| | | DLTs evaluated | subject signs the ICF |
| | | using the NCI | until 28 days after the |
| | | CTCAE criteria, | last dose of study |
| | | version 5.0 | medication. For SAEs made known to |
| | | | the Investigator at any time thereafter |
| | | | that are suspected of being related to |
| | | | study drug, must be reported. For |
| | | | subjects who have high blood pressure |
| | | | related to abiraterone treatment, blood |
| | | | pressure monitoring every 2 weeks |
| | | | should continue after discontinuation of |
| | | | abiraterone until the blood pressure |
| | | | normalizes or returns to pre-enrollment |
| | | | levels |
| | Preliminary | Anti-tumor activity | At pre-specified |
| | efficacy | will be assessed based | timepoints |
| | | on PCWG3 criteria. | |
| Exploratory | PK | Characterize PK of | At pre-specified |
| | endpoints | Compound A in | timepoints |
| | | combination with abiraterone and | |
| | | prednisone | |
| | | Exposure-response relationships | |
| | PD endpoints | Gene expression changes in peripheral | At prespecified timepoints |
| | | blood cell components | |
| | | Gene expression in tumor samples, if | |
| | | available | |
| | | Secreted neuropeptides (such as Pro-GRP, | |
| | | CgA, SYP and NSE) | |
| | | levels in blood | |

AE = Adverse event;
AR = Androgen receptor;
CTC = Circulating tumor cell;
CTCAE = Common Terminology Criteria for Adverse Events;
DLT = Dose-limiting toxicity;
FDG/FDHT = 18-Fluoro-deoxyglucose;
FDHT = 16β-[18F]-Fluoro-5α dihydrotestosterone positron emission tomography;
ICF = Informed consent form;
PCWG3 = Prostate Cancer Clinical Trials Working Group;
PSA = Prostate-specific antigen

Study Design This study will be an open-label, positron emission tomography (PET) imaging proof of biology (POB) study to determine whether Compound A reverses, by the induction of androgen receptor (AR) expression, castration resistance due to lineage switch, in subjects with mCRPC that have failed enzalutamide as last prior therapy. In addition to the up-regulation of neuroendocrine markers, LSD-1 activity may lead to the downregulation of AR levels in prostate tumors. This study aims to assess whether Compound A can induce AR expression and, consequently, re-sensitize tumors to anti-hormonal therapy.

The Screening Period will start 28 days (±3 days) prior to the first dose of Compound A. The informed consent form (ICF) must be signed and dated by the subject and the administering staff prior to the start of any other study-specific procedures. All screening tests and procedures must be completed within the 28 days (±3 days) prior to the first dose of Compound A.

Approximately 10 evaluable subjects will be treated with Compound A monotherapy at 60 mg PO QW, for 4 weeks in Cycle 1. At the end of Cycle 1, subjects will undergo FDG/FDHT PET imaging which will be compared with a Screening assessment to determine the change in AR expression. Subjects must take a minimum of 3 doses of Compound A during Cycle 1 to be considered evaluable. From Cycle 2 onwards, the dose de-escalation is designed to explore lower dose levels of Compound A, 40 mg and 20 mg, in case 60 mg QW is not tolerated in combination with abiraterone, at 1000 mg PO QD, and prednisone, 5 mg PO every 12 hours (10 mg QD) per abiraterone label. The window for the DLT evaluation will be 4 weeks (28 days). Subjects must have taken a minimum of 3 doses of Compound A during the DLT evaluation period to be DLT-evaluable. A Bayesian Interval Dose-Finding Design, modified toxicity probability interval method-2 (mTPI 2) (Guo et al., Contemp Clin Trials. 2017 July; 58:23-33) will be utilized to help guide Compound A dose de-escalation decisions in combination with abiraterone and prednisone. The final dose level decisions will be made by the SRC. A decision table of the optimal decisions (Table 2) is precalculated based on the assumption that the toxicity rate (pT) is lower than or close to a target level 0.3 with the equivalent interval of (0.25, 0.35) to account for the variabilities in the toxicity estimates. At least 3 dose-limiting toxicity (DLT)-evaluable subjects are needed to make a dose de-escalation decision. The dose with an estimated DLT rate closest to 30% and with at least 6 evaluable subjects treated will be determined as a RP2D.

TABLE 2
Decision Table for Target DLT Probability PT = 0.3 with Interval (0.25, 0.35)

| # of DLTs | N = 3 | N = 4 | N = 5 | N = 6 | N = 7 | N = 8 | N = 9 | N = 10 |
|---|---|---|---|---|---|---|---|---|
| 0 | E | E | E | E | E | E | E | E |
| 1 | S | S | E | E | E | E | E | E |
| 2 | D | D | D | S | S | S | E | E |
| 3 | DU | DU | D | D | D | D | S | S |
| 4 | | DU | DU | DU | D | D | D | D |
| 5 | | | DU | DU | DU | DU | DU | D |
| 6 | | | | DU | DU | DU | DU | DU |
| 7 | | | | | DU | DU | DU | DU |
| 8 | | | | | | DU | DU | DU |
| 9 | | | | | | | DU | DU |
| 10 | | | | | | | | DU |

E = Escalate to the next higher dose, however, if the current dose is 60 mg, stay at 60 mg;
S = Stay at the current dose;
D = De-escalate to the next lower dose, however, if the current dose is 20 mg, stop the trial;
DU = De-escalating to the next lower dose and exclude the dose or higher dose from the trial due to unacceptable toxicity;
DLT = dose limiting toxicity;

After Cycle 1 and Cycle 3, subjects will undergo FDG/FDHT PET imaging will be compared with Screening and to determine the change in AR expression, established according to local MSKCC definition based on PET imaging analysis. From Cycle 2 onwards, subjects will be followed up based on PCWG3 criteria. Biopsies will be obtained, whenever safe and feasible, to perform PD analyses pre- and on-treatment. Study treatment may be discontinued if there is evidence of disease progression, unacceptable toxicity or subject/physician decision to withdraw.

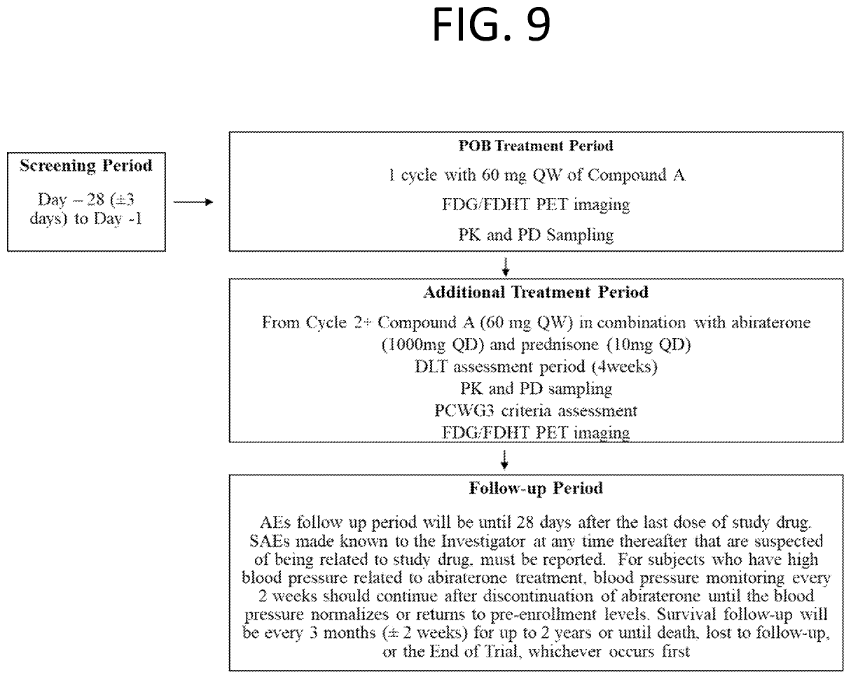

FIG. 9 shows the overall study design, which is also shown below.

Study Population/Number of Subjects This will be a single center, open-label study in which approximately 10 evaluable subjects will be enrolled.

Inclusion Criteria Subjects will satisfy the following criteria to be enrolled in the study: (1) Subject is a male ≥18 years of age at the time of signing the informed consent form (ICF). (2) Histologically confirmed adenocarcinoma of the prostate. (3) Surgically or medically castrated, with testosterone levels of ≤50 ng/dL (<2.0 nM). If the subject is being treated with luteinizing hormone-releasing hormone (LHRH) agonists/antagonists (subject who have not undergone orchiectomy) this therapy must have been initiated at least 4 weeks prior to Cycle 1 Day 1 and must be continued throughout the study. (4) Subjects must have failed prior therapy with enzalutamide (a) Has completed at least 12 weeks of prior continuous therapy with enzalutamide; and (b) Has not been without enzalutamide treatment for >15 days prior to initiation of study treatment. (5) Subjects who have received abiraterone prior to enzalutamide are eligible. (6) One to two line(s) of prior taxane-based chemotherapy are allowed. If docetaxel chemotherapy is used more than once, this will be considered as one regimen. (7) Documented prostate cancer progression as assessed by the investigator with one of the following: (a) PSA progression defined by a minimum of 3 rising PSA levels with an interval of ≥1 week between each determination. The PSA value at screening must be ≥1 μg/L (1 ng/mL) if PSA is the only indication of progression; subjects on systemic glucorticoids for control of symptoms must have documented PSA progression by PCWG3 criteria while on systemic glucocorticoids prior to commencing Cycle 1 Day 1 treatment; and (b) Radiographic progression of soft tissue disease by RECIST 1.1 or bone metastasis with 2 or more documented new bone lesions on a bone scan with or without PSA progression. (8) Subjects must have FDHT lesion >2 cm lesion that has an SUVₘₐₓ of 2.9 or less in bone, or 2.4 or less in soft tissue, or two or more smaller lesions that meet those criteria. (9) Eastern Cooperative Oncology Group (ECOG) performance status of 0 or 1 at Screening. (10) Subjects must have the following laboratory values: (a) Absolute neutrophil count (ANC)≥1.5×109/L without growth factor support for 7 days (14 days if subject received pegfilgastrim); (b) Hemoglobin (Hgb)≥9 g/dL (≥90 g/L or >5.59 mmol/L); (c) Platelet count (plt)≥100×10⁹/L; (d) Serum potassium concentration within normal range, or correctable with supplements; (e) Serum AST/SGOT and ALT/SGPT≤3.0×Upper Limit of Normal (ULN) or ≤5.0×ULN if liver metastases are present; (f) Serum total bilirubin ≤1.5×ULN (≤2×ULN in case of documentened Gilbert); (g) Subjects must have serum albumin ≥3.0 g/dL; (h) Serum creatinine ≤1.5×ULN, or measured glomerular filtration rate (GFR)≥60 mL/min/1.73 m² using an exogenous filtration marker such as iohexol, inulin, 51Cr EDTA or ¹²⁵I iothalamate. In cases where the serum creatinine is <1.5×ULN, there is no need to calculate GFR; (i) PT (or INR) and activated partial thromboplastin time (APTT) (i) within normal range and ≤1.5 ULN.

Screening Period The Screening window starts 28 days (±3 days) prior to the first dose of Compound A. Waivers to the protocol will not be granted during the conduct of this trial, under any circumstances. Safety laboratory analyses will be performed locally. Screening laboratory values must demonstrate subject eligibility, but may be repeated within the screening window, if necessary.

Investigation Products (Abiraterone) Abiraterone (trade name Zytiga™) is a 250 mg white to off-white tablet.

Abiraterone will be prescribed, sourced and administered locally as standard of care. Management (ie, handling, storage, administration, and disposal) of abiraterone will be in accordance with the relevant local guidelines and package insert.

Investigation Products (Prednisone) This description is for the 2 mg tablet. Prednisone is a yellowish-white tablet.

Prednisone will be prescribed, sourced and administered locally as standard of care. Management (ie, handling, storage, administration, and disposal) of prednisone will be in accordance with the relevant local guidelines and package insert.

Treatment Administration and Schedule Compound A will be administered PO QW at 60 mg in a 4-week Cycle. Compound A will be administered with at least 240 mL of water. Subjects should fast for a minimum of 4 hours prior to Compound A administration and refrain from any food intake for up to 1 hour after dosing.

Since the combination with abiraterone and prednisone has not been tested before, a Bayesian Interval Dose-Finding Design, modified toxicity probability interval method-2 (mTPI-2) (Guo, Contemp Clin Trials. 2017 July; 58:23-33) will be utilized to help guide Compound A dose de-escalation to a lower dose level, 40 mg and 20 mg, in the case that 60 mg of Compound A is not tolerated in combination with abiraterone and prednisone with the final decisions being made by the SRC.

On study days that require PK assessments, Compound A will be administered in the clinic after any pre-dose assessments are completed. On all other study days, subjects will self-administer their assigned doses at home, and record dosing times and fasting period.

Study treatment may be discontinued if there is evidence of clinically significant disease progression, unacceptable toxicity or subject/physician decision to withdraw.

The recommended dose for abiraterone is 1000 mg (four 250 mg tablets) PO QD that must not be taken with food (Table 3). The tablets should be taken at least two hours after eating and no food should be eaten for at least one hour after taking the tablets. These should be swallowed whole with water. Taking the tablets with food increases systemic exposure to abiraterone.

Prednisone dose is 5 mg PO every 12 hours (10 mg QD) (Table 3). The administration will begin with the first day of abiraterone and continuing until the discontinuation of abiraterone.

TABLE 3
Table of dose administration of Compound A, abiraterone and prednisone

| Drug | Dose | Route | days | Notes |
|---|---|---|---|---|
| Compound A | 60 mg | PO | 1, 8, 15, 22 | From cycle 1 |
| Abiraterone | 1000 mg | PC | QD | From cycle ≥2 |
| Prednisone | 10 mg | PO | QD | From cycle ≥2 |

PO = administered orally;
QD = daily

Primary and Efficacy Analysis Efficacy analyses will be based on the evaluable population. The primary endpoint for proof of biology is percentage change of AR level, assessed using FDG/FDHT PET imaging, at week 4 for the monotherapy period and at week 12 from Screening for the combined therapy period of Compound A with Abiraterone and Prednisone. Additional efficacy endpoint to be analyzed include the Objective Soft Tissue Response, Overall response rate (ORR) (defined as the percentage of subjects whose best response is complete response or partial response), the radiographic progression-free survival (rPFS) (defined as the time from the first dose of Compound A to the first objective evidence of radiographic progression or death from any cause, whichever occurs first, for all treated subjects with soft tissue and/or bone disease as the time from first dose of Compound A) and progression-free survival (PFS) (defined as the time from the first dose of study drug to the first occurrence of disease progression or death from any cause, whichever occurs first) assessed by investigators according to PCWG3 criteria for the combination of Compound A with abiraterone and prednisone. Primary and efficacy endpoints will be analyzed descriptively based on treated subjects. Point estimates and 2-sided 95% exact Clopper-Pearson confidence intervals (CIs) will be reported.

Objective Soft Tissue Response: The objective soft tissue response rate is defined as the proportion of subjects who achieve a best response of partial response or better (PR or CR) per PCWG3 criteria. Analysis of objective soft tissue response will be based on the treated population and the evaluable population who have soft tissue disease. The number and percentage of subjects in the following response categories will be presented: partial response (PR), complete response (CR), overall response (CR+PR), stable disease (SD), progressive disease (PD), and not evaluable (NE).

The corresponding 95% exact Clopper-Pearson CI for each response category will also be provided.

Duration of Response: Duration of response for soft tissue disease is defined as the time from the earliest date of documented soft tissue response (PR or CR PCWG3 criteria) to the first documented soft tissue disease progression or death, whichever occurs first. Duration of response will be summarized using Kaplan-Meier estimates by dose level. The analysis population is confined to those who have responded. Subjects who neither progress nor die by a data cutoff date will be censored at the date of their last adequate soft tissue tumor assessment.

Proportion of Subjects Alive and Not Progressed: The proportion of subjects alive and not progressed is defined as the proportion of subjects alive who have not progressed at 6 months follow-up with progression defined per PCWG3 criteria. The proportion of subjects alive and not progressed at 6 months will be estimated using the Kaplan-Meier method for the treated population.

Progression-free Survival: The duration of PFS will be calculated for all treated subjects defined as the time from the first dose of study drug to the first occurrence of disease progression or death from any cause, whichever occurs first. Disease progression is defined as progressive disease by PCWG3. Conventions for censoring will be described in the statistical analysis plan (SAP). The PFS will be estimated using the Kaplan-Meier method for the treated population.

Radiographic Progression free Survival: The duration of rPFS will be calculated for all treated subjects with soft tissue and/or bone disease as the time from the first dose of Compound A to the first objective evidence of radiographic progression or death from any cause, whichever occurs first. Radiographic disease progression is defined as progressive disease by PCWG3. Conventions for censoring will be described in the statistical analysis plan (SAP). The rPFS will be estimated using the Kaplan-Meier method for the treated population.

Overall Survival: Overall Survival (OS) is defined as the time from the first dose of Compound A to death from any cause. Subjects who are still alive at the clinical cut-off date for the analysis will be censored at the last known alive date. The OS rate at 12 months and at 24 months will be summarized using the Kaplan-Meier method for the treated population.

While certain embodiments have been illustrated and described, it should be understood that changes and modifications can be made therein in accordance with ordinary skill in the art without departing from the technology in its broader aspects as defined in the following claims.

1. A method of treating metastatic castration-resistant prostate cancer (mCRPC) in a subject in need thereof comprising administering to the subject a besylate salt of an LSD-1 inhibitor compound having the structure:

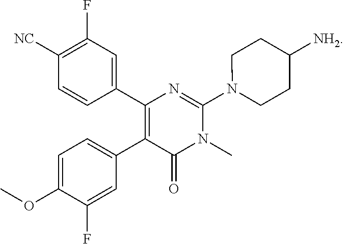 [SMILES: COc1ccc(-c2c(-c3ccc(C#N)c(F)c3)nc(N3CCC(N)CC3)n(C)c2=O)cc1F]

2. The method of claim 1, wherein in a first 28-day cycle, the LSD-1 inhibitor is administered:
(a) orally; and/or
(b) at the dose of about 60 mg; and/or
(c) once a week; and/or
(d) on Days 1, 8, 15 and 22 of a 28 day cycle.

3. The method of claim 1, wherein the subject has failed prior androgen receptor pathway inhibitor therapy.

4. The method of claim 1, wherein the prior androgen receptor pathway inhibitor therapy is enzalutamide.

5. The method of claim 1, further comprising the step of administering an androgen receptor pathway inhibitor and prednisone in combination with the LSD-1 inhibitor.

6. The method of claim 5, wherein the subject has failed prior androgen receptor pathway inhibitor therapy and the androgen receptor pathway inhibitor is either different or the same as the prior androgen receptor pathway inhibitor.

7. The method of claim 5, wherein the prior androgen receptor pathway inhibitor therapy is enzalutamide.

8. The method of claim 6, wherein the androgen receptor pathway inhibitor is abiraterone.

9. The method of claim 8, wherein the abiraterone is administered:
(a) orally; and/or
(b) at the dose of about 1000 mg; and/or
(c) once daily; and/or
(d) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

10. The method of claim 5, wherein the prednisone is administered:
(a) orally; and/or
(b) at a dose of about 5 mg; and/or
(c) every 12 hours; and/or
(d) twice daily; and/or
(e) at a total dose of about 10 mg per day;
(f) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

11. The method of claim 1, wherein the LSD-1 inhibitor reverses the castration resistance due to lineage switch.

12. The method of claim 11, further comprising the step of administering an androgen receptor pathway inhibitor and prednisone in combination with the LSD-1 inhibitor.

13. The method of claim 11, wherein the subject has failed prior androgen receptor pathway inhibitor therapy and the androgen receptor pathway inhibitor is either different or the same as the prior androgen receptor pathway inhibitor.

14. The method of claim 11, wherein the prior androgen receptor pathway inhibitor therapy is enzalutamide.

15. The method of claim 12, wherein the androgen receptor pathway inhibitor is abiraterone.

16. The method of claim 15, wherein the abiraterone is administered:
(a) orally; and/or
(b) at the dose of about 1000 mg; and/or
(c) once daily; and/or
(d) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

17. The method of claim 12, wherein the prednisone is administered:
(a) orally; and/or
(b) at a dose of about 5 mg; and/or
(c) every 12 hours; and/or
(d) twice daily; and/or
(e) at a total dose of about 10 mg per day;
(f) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

18. A method of treating metastatic castration-resistant prostate cancer (mCRPC) in a subject who has failed prior therapy with enzalutimide comprising:
(a) a first step of administering to the subject a besylate salt of an LSD-1 inhibitor compound having the structure: 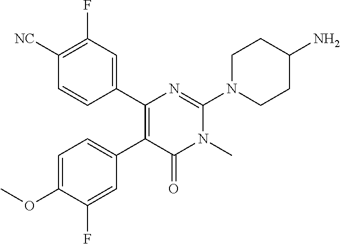 [SMILES: COc1ccc(-c2c(-c3ccc(C#N)c(F)c3)nc(N3CCC(N)CC3)n(C)c2=O)cc1F]
in a 28-day cycle:
(i) orally; and/or
(ii) at the dose of about 60 mg; and/or
(iii) once a week; and/or
(iv) on Days 1, 8, 15 and 22 of a 28 day cycle,
wherein the LSD-1 inhibitor reverses the castration resistance due to lineage switch, thereafter
(b) a second step of concomitantly administering the LSD-1 inhibitor, abiraterone, and prednisone, wherein the LSD-1 inhibitor is administered in a 28-day cycle:
(i) orally; and/or
(ii) at the dose of about 60 mg, 40 mg, or 20 mg; and/or
(iii) once a week; and/or
(iv) on Days 1, 8, 15 and 22 of a 28 day cycle,
wherein the abiraterone is administered:
(i) orally; and/or
(ii) at the dose of about 1000 mg; and/or
(iii) once daily; and/or
(iv) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle, and
wherein the prednisone is administered:
(i) orally; and/or
(ii) at a dose of about 5 mg; and/or
(iii) every 12 hours; and/or
(iv) twice daily; and/or
(v) at a total dose of about 10 mg per day;
(vi) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.

19. The method of claim 18, wherein the tumor has reduced in size, or PSA levels have decreased compared to baseline, or a combination thereof.

20. A pharmaceutical composition for use in treating metastatic castration-resistant prostate cancer (mCRPC) in a subject in need thereof, wherein the composition comprises a besylate salt of an LSD-1 inhibitor compound having the structure:

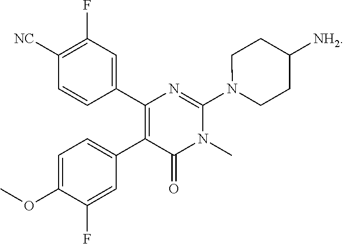 [SMILES: COc1ccc(-c2c(-c3ccc(C#N)c(F)c3)nc(N3CCC(N)CC3)n(C)c2=O)cc1F]

21. The composition of claim 20, for use in a method of treating metastatic castration-resistant prostate cancer (mCRPC) in a subject in need thereof, wherein the method comprises administering the composition, and wherein in a first 28-day cycle, the LSD-1 inhibitor is administered:
(a) orally; and/or
(b) at the dose of about 60 mg; and/or
(c) once a week; and/or
(d) on Days 1, 8, 15 and 22 of a 28 day cycle.

22. A pharmaceutical composition for use in a method of treating metastatic castration-resistant prostate cancer (mCRPC) in a subject who has failed prior therapy with enzalutamide, wherein the composition comprises a besylate salt of an LSD-1 inhibitor compound having the structure: 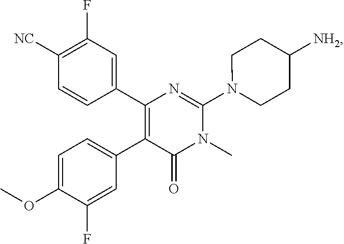 [SMILES: COc1ccc(-c2c(-c3ccc(C#N)c(F)c3)nc(N3CCC(N)CC3)n(C)c2=O)cc1F]
wherein the method comprises a first step of administering the pharmaceutical composition to the subject in a 28-day cycle:
(i) orally; and/or
(ii) at the dose of about 60 mg; and/or
(iii) once a week; and/or
(iv) on Days 1, 8, 15 and 22 of a 28 day cycle,
wherein the LSD-1 inhibitor reverses the castration resistance due to lineage switch, and
wherein thereafter the method comprises a second step of concomitantly administering the LSD-1 inhibitor, abiraterone, and prednisone, wherein the LSD-1 inhibitor is administered in a 28-day cycle:
(i) orally; and/or
(ii) at the dose of about 60 mg, 40 mg, or 20 mg; and/or
(iii) once a week; and/or
(iv) on Days 1, 8, 15 and 22 of a 28 day cycle,
wherein the abiraterone is administered:
(i) orally; and/or
(ii) at the dose of about 1000 mg; and/or
(iii) once daily; and/or
(iv) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle, and
wherein the prednisone is administered:
(i) orally; and/or
(ii) at a dose of about 5 mg; and/or
(iii) every 12 hours; and/or
(iv) twice daily; and/or
(v) at a total dose of about 10 mg per day;
(vi) on Days 1, 2, 3, 4, 5, 6, 7, 8, 9, 10, 11, 12, 13, 14, 15, 16, 17, 18, 19, 20, 21, 22, 23, 24, 25, 26, 27, or 28 of a 28 day cycle.
